(19) United States
(12) Patent Application Publication
(10) Pub. No.: US 2008/0103122 A1
(43) Pub. Date: May 1, 2008

(54) PHARMACEUTICAL COMBINATIONS FOR LIPID MANAGEMENT AND IN THE TREATMENT OF ATHEROSCLEROSIS AND HEPATIC STEATOSIS

(75) Inventor: Enrico P. Veltri, Princeton, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 11/846,575

(22) Filed: Aug. 29, 2007

Related U.S. Application Data
(60) Provisional application No. 60/842,211, filed on Sep. 5, 2006.

(51) Int. Cl. A61K 31/397; A61K 31/497; A61P 7/00

(52) U.S. Cl. 514/210.02; 514/252.14

(57) ABSTRACT

A pharmaceutical combination comprising an effective amount of at least one cholesterol absorption inhibitor and at least one microsomal triglyceride transfer protein inhibitor (MTP).

### RELATED APPLICATIONS

This application claims priority to provisional application U.S. Ser. No. 60/842,211, filed on Sep. 5, 2006, herein incorporated by reference.

### FIELD OF THE INVENTION

The present invention relates to pharmaceutical combinations which are used in lipid management of a mammal, such as a human, and in the treatment of atherosclerosis and hepatic steatosis by administering an effective amount of the pharmaceutical combination. The pharmaceutical combinations comprise at least one cholesterol absorption inhibitor (CAI) and a microsomal triglyceride transfer protein (MTP) inhibitor.

### BACKGROUND OF THE INVENTION

Vascular disease is a term which broadly encompasses all disorders of blood vessels including small and large arteries and veins and blood flow. The most prevalent form of vascular disease is arteriosclerosis, a condition associated with the thickening and hardening of the arterial wall. Arteriosclerosis of the large vessels is referred to as atherosclerosis. Atherosclerosis is the predominant underlying factor in vascular disorders such as coronary artery disease, aortic aneurysm, arterial disease of the lower extremities and cerebrovascular disease.

One major risk factor for arteriosclerosis is high serum cholesterol. A total cholesterol level in excess of 225-250 mg/dl is associated with significant elevation of risk of vascular disease, particularly coronary heart disease.

Cholesteryl esters are a major component of atherosclerotic lesions and the major storage form of cholesterol in arterial wall cells. Formation of cholesteryl esters is also a step in the intestinal absorption of dietary cholesterol. Thus, inhibition of cholesteryl ester formation and reduction of serum cholesterol can inhibit the progression of atherosclerotic lesion formation, decrease the accumulation of cholesteryl esters in the arterial wall, and block the intestinal absorption of dietary cholesterol.

The regulation of whole-body cholesterol homeostasis in mammals and animals involves the regulation of dietary cholesterol and modulation of cholesterol biosynthesis, bile acid biosynthesis and the catabolism of the cholesterol-containing plasma lipoproteins. The liver is the major organ responsible for cholesterol biosynthesis and catabolism and, for this reason, it is a prime determinant of plasma cholesterol levels. The liver is the site of synthesis and secretion of very low density lipoproteins (VLDL) which are subsequently metabolized to low density lipoproteins (LDL) in the circulation. LDL are the predominant cholesterol-carrying lipoproteins in the plasma and an increase in their concentration is correlated with increased atherosclerosis. When intestinal cholesterol absorption is reduced, by whatever means, less cholesterol is delivered to the liver. The consequence of this action is decreased hepatic lipoprotein (VLDL) production and an increase in the hepatic clearance of plasma cholesterol, mostly as LDL. Thus, the net effect of inhibiting intestinal cholesterol absorption is a decrease in plasma cholesterol levels.

U.S. Pat. Nos. 5,846,966 and 5,661,145, respectively, disclose treatments for inhibiting atherosclerosis and reducing plasma cholesterol levels using such hydroxy-substituted azetidinone compounds or substituted β-lactam compounds in combination with HMG-CoA reductase inhibitor compounds, which act by blocking hydroxymethylglutaryl coenzyme A (HMG-CoA) reductase (the rate-limiting enzyme in hepatic cholesterol synthesis). HMG-CoA reductase inhibitors, e.g., statins such as lovastatin, simvastatin, and pravastatin, slow the progression of atherosclerotic lesions in the coronary and carotid arteries. Simvastatin and pravastatin have also been shown to reduce the risk of coronary heart disease events in patients with hypercholesterolemia and/or atherosclerotic coronary heart disease (CHD).

Simvastatin is marketed worldwide, and sold in the U.S. under the tradename ZOCOR®. Methods for making it are described in U.S. Pat. Nos. 4,444,784; 4,916,239; 4,820,850; among other patent and literature publications.

U.S. Pat. No. 5,698,527 discloses ergostanone derivatives substituted with disaccharides as cholesterol absorption inhibitors, employed alone or in combination with certain other cholesterol lowering agents, which are useful in the treatment of hypercholesterolemia and related disorders.

Other vascular conditions frequently coexist with cholesterol levels associated with atherosclerosis. These may include hypertension, angina and/or arrhythmia. The relevance of, for example, elevated blood pressure as a risk factor for atherosclerosis, cardiovascular and cerebrovascular disease in both men and women has been clarified in a large number of epidemiological studies.

Clinical trials of blood pressure lowering using cardiovascular agents including, for example, calcium channel blockers, have shown beneficial effects in the treatment of early atherosclerotic lesions (see, e.g., Lichtien, P. R. et al.: Lancet, 335: 1109-1113 (1990) and Waters, D. et al. Circulation 82: 1940-1953 (1990)). Scott (PCT patent Application No. WO 99/11260) describes combinations of an HMG CoA reductase inhibitor with an antihypertensive agent for the treatment of atherosclerosis and other symptoms of vascular disease risk. Additionally, Egon et al. (PCT Patent Application No. WO 96/40255) describe a combination therapy of antihypertensive agents including eplerenone and angiotensin II antagonist for treating cardiovascular disease.

In vitro MTP catalyzes the transport of lipid molecules between phospholipid membranes. See, U.S. Pat. No. 6,472,414 B1. In vivo it has been reported that MTP mediates trigyceride absorption and chylomicron secretion from the intestine and VLDL secretion from the liver, by linking lipid molecules with apolipoprotein B (ApoB). (See, abstract of S. Williams & J. D. Best, Expert Opinion on Therapeutic Patents (April 2003, vol. 13, no. 4, pp. 470-488), www.expertopin.com/doi/abs/10.1517/13543776.13.4.479 ?cookieSet+1&journalCode). It follows that inhibition of MTP could reduce the level of all ApoB-containing proteins, including LDL. Drugs that inhibit MTP, therefore, potentially could be effective in reducing atherosclerotic vascular disease by lowering all levels of atherogenic lipoproteins. One commentator has suggested that while partial inhibition of MTP by an inhibitor could be useful when combined with other drugs that alter lipid metabolism, marked inhibition of MTO could cause significant adverse effects (Williams & Best).

Substances that inhibit MTP are well known in the art. See US 2006/0166999 A1 and U.S. Pat. No. 6,472,414 B2, both herein incorporated by reference, which cites to EP 705 831, EP 779 279, EP 779 276, EP 802 198 and EP 799 828, also incorporated by reference. Zaiss et al., Circulation, 100 (18 Suppl. I): 255 Abst. 13423 (1999) reports that implitiapide, a MTP inhibitor, prevents the formation of atherosclerotic plaques in mice.

WO 2005/087234 A1, incorporated by reference, discloses method and compositions for treating hyperlipidemia and/or hypercholesterolemia that comprise administering to the subject and effective amount of an MTP inhibitor, wherein said administration comprises at least three step-wise, increasing dosages of the MTP inhibitor; the MTP inhibitor may be combined with a further lipid modifying compound, such as a HMG Co-A reductase inhibitor or ezetimibe.

WO 00/38725 A1, incorporated by reference, discloses cardiovascular therapeutic combinations including an ileal bile acid transport inhibitor or cholesteryl ester transport protein inhibitor in combination with a fibric acid derivative, nicotinic acid derivative, microsomal triglyceride transfer protein inhibitor, cholesterol absorption antagonist, phytosterol, stanol, antihypertensive agent or bile acid sequestrant.

Despite recent improvements in the management of lipid levels in mammals, such as humans, as well as for the treatment for atherosclerosis, hyperlipidemia, lyperlipenia, hypertriglyceridemia, other vascular diseases and hepatic steatosis, there remains a need in the art for improved compositions and treatments these disease states.

### SUMMARY OF THE INVENTION

The present invention provides for pharmaceutical combinations comprising at least one cholesterol absorption inhibitor, e.g., a sterol absorption inhibitor or a 5-α-stanol absorption inhibitor, and at least one microsomal triglyceride transfer protein inhibitor.

The present invention also provides for a method for lipid management in a mammal in need thereof which comprises administering an effective amount of a pharmaceutical combination comprising at least one cholesterol absorption inhibitor, e.g., a sterol absorption inhibitor or a 5-α-stanol absorption inhibitor, and at least one microsomal triglyceride transfer protein inhibitor.

An alternative embodiment of the present invention also provides for a method for the treatment, prevention or ameliorating the symptoms atherosclerosis in a mammal in need thereof by administering an effective amount of a composition comprising at least one cholesterol absorption inhibitor, e.g., a sterol absorption inhibitor, a 5-α-stanol absorption inhibitor, and at least one microsomal triglyceride transfer protein inhibitor.

Another embodiment of this invention provides for the mitigation, prevention or amelioration the symptoms or development of hepatic steatosis in a mammal in need thereof by administering at least one cholesterol absorption inhibitor, e.g., a sterol absorption inhibitor or a 5-α-stanol absorption inhibitor, and at least one microsomal triglyceride transfer protein inhibitor.

Another embodiment of the present invention also provides for a method for lipid management in a mammal in need thereof which comprises administering an effective amount of a pharmaceutical combination comprising at least one cholesterol absorption inhibitor, e.g., a sterol absorption inhibitor or a 5-α-stanol absorption inhibitor, at least one microsomal triglyceride transfer protein inhibitor and at least one cholesterol lowering agent, e.g., a HMG-CoA reductase inhibitor.

A further embodiment of the present invention provides for a method for the treatment, prevention or ameliorating the symptoms atherosclerosis in a mammal in need thereof by administering an effective amount of a composition comprising at least one cholesterol absorption inhibitor, e.g., a sterol absorption inhibitor, or a 5-α-stanol absorption inhibitor, at least one microsomal triglyceride transfer protein inhibitor and at least one cholesterol lowering agent, e.g., a HMG-CoA reductase inhibitor.

Another embodiment of this invention provides for the mitigation, prevention or amelioration the symptoms or development of hepatic steatosis in a mammal in need thereof by administering at least one cholesterol absorption inhibitor, e.g., a sterol absorption inhibitor or a 5-α-stanol absorption inhibitor, at least one microsomal triglyceride transfer protein inhibitor, and at least one cholesterol lowering agent, e.g., a HMG-CoA reductase inhibitor.

The present invention also relates to a kit for lipid management in a mammal or for the treatment, prevention or amelioration of the symptoms of atherosclerosis or hepatic steatosis which comprises at least one cholesterol absorption inhibitor and at least one microsomal triglyceride transfer protein inhibitor in separate form.

### DETAILED DESCRIPTION

The terms used herein have their ordinary meaning and the meaning of such terms is independent at each occurrence thereof. That notwithstanding and except where stated otherwise, the following definitions apply throughout the specification and claims: Chemical names, common names and chemical structures may be used interchangeably to describe that same structure. These definitions apply regardless of whether a term is used by itself or in combination with other terms, unless otherwise indicated Hence, the definition of “alkyl” applies to “alkyl” as well as the “alkyl” portion of “hydroxyalkyl”, “haloalkyl”, “alkoxy” etc.

As used above, and throughout the specification, the following terms, unless otherwise indicated, shall be understood to have the following meanings:

“Patient” includes both human and animals.

“Mammal” means humans and other mammalian animals.

“Alkyl” means an aliphatic hydrocarbon group which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more lower alkyl groups such as methyl, ethyl or propyl, are attached to a linear alkyl chain. “Lower alkyl” means a group having about 1 to about 6 carbon atoms in the chain which may be straight or branched. Non-limiting examples of suitable alkyl groups include methyl, ethyl, n-propyl, isopropyl, n-butyl, t-butyl, n-pentyl, heptyl, nonyl and decyl. R³²-substituted alkyl groups include fluoromethyl, trifluoromethyl and cyclopropylmethyl.

“Alkenyl” means an aliphatic hydrocarbon group containing at least one carbon-carbon double bond and which may be straight or branched and comprising about 2 to about 15 carbon atoms in the chain. Preferred alkenyl groups have about 2 to about 12 carbon atoms in the chain; and more preferably about 2 to about 6 carbon atoms in the chain. Branched means that one or more lower alkyl groups such as methyl, ethyl or propyl, are attached to a linear alkenyl chain. “Lower alkenyl” means about 2 to about 6 carbon atoms in the chain which may be straight or branched. Non-limiting examples of suitable alkenyl groups include ethenyl, propenyl, n-butenyl, 3-methylbut-2-enyl, n-pentenyl, octenyl and decenyl.

“Alkynyl” means an aliphatic hydrocarbon group containing at least one carbon-carbon triple bond and which may be straight or branched and comprising about 2 to about 15 carbon atoms in the chain. Preferred alkynyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain. “Lower alkynyl” means about 2 to about 6 carbon atoms in the chain which may be straight or branched, Non-limiting examples of suitable alkynyl groups include ethynyl, propynyl, 2-butynyl, 3-methylbutynyl, n-pentynyl, and decynyl.

“Aryl” means an aromatic monocyclic or multicyclic ring system comprising about 6 to about 14 carbon atoms, preferably about 6 to about 10 carbon atoms. The aryl group can be optionally substituted with one or more substituents, which may be the same or different, and are as defined herein or two substituents on adjacent carbons can be linked together to form 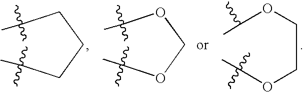
Non-limiting examples of suitable aryl groups include phenyl and naphthyl.

“Heteroaryl” means an aromatic monocyclic or multicyclic ring system comprising about 5 to about 14 ring atoms, preferably about 5 to about 10 ring atoms, in which one to four of the ring atoms is an element other than carbon, for example nitrogen, oxygen or sulfur, alone or in combination. Preferred heteroaryls contain about 5 to about 6 ring atoms. The “heteroaryl” can be optionally substituted by one or more substituents, which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. Non-limiting examples of suitable heteroaryls include pyridyl, pyrazinyl, furanyl, thienyl, pyrimidinyl, isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like.

“Cycloalkyl” means a non-aromatic mono- or multicyclic ring system comprising about 3 to about 10 carbon atoms, preferably about 5 to about 10 carbon atoms. Preferred cycloalkyl rings contain about 5 to about 7 ring atoms. The cycloalkyl can be optionally substituted with one or more substituents which may be the same or different, and are as defined above. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalin, norbornyl, adamantyl and the like. Further non-limiting examples of cycloalkyl include the following:

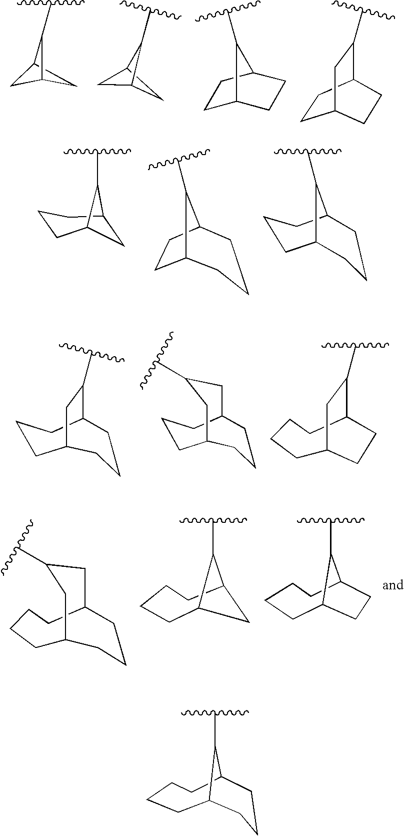

“Cycloalkylether” means a non-aromatic ring of 3 to 7 members comprising an oxygen atom and 2 to 7 carbon atoms. Ring carbon atoms can be substituted, provided that substituents adjacent to the ring oxygen do not include halo or substituents joined to the ring through an oxygen, nitrogen or sulfur atom.

“Cycloalkenyl” means a non-aromatic mono or multicyclic ring system comprising about 3 to about 10 carbon atoms, preferably about 5 to about 10 carbon atoms which contains at least one carbon-carbon double bond. The cycloalkenyl ring can be optionally substituted with one or more substituents which may be the same or different, and are as defined above. Preferred cycloalkenyl rings contain about 5 to about 7 ring atoms. Non-limiting examples of suitable monocyclic cycloalkenyls include cyclopentenyl, cyclohexenyl, cycloheptenyl, and the like, Non-limiting example of a suitable multicyclic cycloalkenyl is norbornylenyl.

“Heterocyclenyl” (or “heterocycloalkeneyl”) means a non-aromatic monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur atom, alone or in combination, and which contains at least one carbon-carbon double bond or carbon-nitrogen double bond. There are no adjacent oxygen and/or sulfur atoms present in the ring system. Preferred heterocyclenyl rings contain about 5 to about 6 ring atoms. The prefix aza, oxa or thia before the heterocyclenyl root name means that at least a nitrogen, oxygen or sulfur atom respectively is present as a ring atom. The heterocyclenyl can be optionally substituted by one or more substituents. The nitrogen or sulfur atom of the heterocyclenyl can be optionally oxidized to the corresponding N-oxide, S-oxide or S,S-dioxide. Non-limiting examples of suitable monocyclic azaheterocyclenyl groups include 1,2,3,4-tetrahydropyridyl, 1,2-dihydropyridyl, 1,4-dihydropyridyl, 1,2,3,6-tetrahydropyridyl, 1,4,5,6-tetrahydropyrimidyl, 2-pyrrolinyl, 3-pyrrolinyl, 2-imidazolinyl, 2-pyrazolinyl, and the like. Non-limiting examples of suitable oxaheterocyclenyl groups include 3,4-dihydro-2H-pyran, dihydrofuranyl, fluorodihydrofuranyl, and the like. Non-limiting example of a suitable multicyclic oxaheterocyclenyl group is 7-oxabicyclo[2.2.1]heptenyl. Non-limiting examples of suitable monocyclic thiaheterocyclenyl rings include dihydrothiophenyl, dihydrothiopyranyl, and the like.

“Halo” means fluoro, chloro, bromo, or iodo groups. Preferred are fluoro, chloro or bromo, and more preferred are fluoro and chloro.

“Haloalkyl” means an alkyl as defined above wherein one or more hydrogen atoms on the alkyl is replaced by a halo group defined above.

“Heterocyclyl” (or heterocycloalkyl) means a non-aromatic saturated monocyclic or multicyclic ring system comprising about 3 to about 10 ring atoms, preferably about 5 to about 10 ring atoms, in which 1-3, preferably 1 or 2 of the atoms in the ring system is an element other than carbon, for example nitrogen, oxygen or sulfur, alone or in combination. There are no adjacent oxygen and/or sulfur atoms present in the ring system. Preferred heterocyclyls contain about 5 to about 6 ring atoms. The prefix aza, oxa or thia before the heterocyclyl root name means that at least a nitrogen, oxygen or sulfur atom respectively is present as a ring atom. The heterocyclyl can be optionally substituted by one or more which may be the same or different, and are as defined herein. The nitrogen or sulfur atom of the heterocyclyl can be optionally oxidized to the corresponding N-oxide, S-oxide or S,S-dioxide. Non-limiting examples of suitable monocyclic heterocyclyl rings include piperidyl, pyrrolidinyl, piperazinyl, morpholinyl, thiomorpholinyl, thiazolidinyl, 1,3-dioxolanyl, 1,4-dioxanyl, tetrahydrofuranyl, tetrahydrothiophenyl, tetrahydrothiopyranyl, and the like.

“Arylalkyl” means an aryl-alkyl- group in which the aryl and alkyl are as previously described. Preferred aralkyls comprise a lower alkyl group. Non-limiting examples of suitable aralkyl groups include benzyl, 2-phenethyl and naphthalenylmethyl. The bond to the parent moiety is through the alkyl.

“Arylcycloalkyl” means a group derived from a fused aryl and cycloalkyl as defined herein. Preferred arylcycloalkyls are those wherein aryl is phenyl and cycloalkyl consists of about 5 to about 6 ring atoms. The arylcycloalkyl can be optionally substituted by one or more substituents. Non-limiting examples of suitable arylcycloalkyls include indanyl and 1,2,3,4-tetrahydronaphthyl and the like. The bond to the parent moiety is through a non-aromatic carbon atom.

“Arylheterocycloalkyl” means a group derived from a fused aryl and heterocycloalkyl as defined herein. Preferred arylcycloalkyls are those wherein aryl is phenyl and heterocycloalkyl consists of about 5 to about 6 ring atoms. The arylheterocycloalkyl can be optionally substituted by one or more substituents. Non-limiting examples of suitable arylheterocycloalkyls include

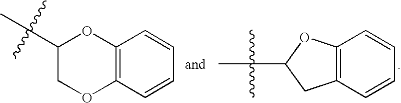

The bond to the parent moiety is through a non-aromatic carbon atom.

“Acyl” means an organic group in which the —OH of the carboxyl group is replaced by some other substituent. Suitable non-limiting examples include H—C(O)—, alkyl-C(O)—, alkenyl-C(O)—, alkynyl-C(O)—, aryl-C(O)— or cycloalkyl-C(O)— group in which the various groups are as previously described. The bond to the parent moiety is through the carbonyl. Preferred acyls contain a lower alkyl. Non-limiting examples of suitable acyl groups include formyl, acetyl, propanoyl, 2-methylpropanoyl, butanoyl and cyclohexanoyl.

“Alkoxy” means an alkyl-O— group in which the alkyl group is as previously described. Non-limiting examples of suitable alkoxy groups include methoxy, ethoxy, n-propoxy, isopropoxy, n-butoxy and heptoxy. The bond to the parent moiety is through the ether oxygen.

“Alkyoxyalkyl” means a group derived from an alkoxy and alkyl as defined herein. The bond to the parent moiety is through the alkyl.

“Arylalkenyl” means a group derived from an aryl and alkenyl as defined herein. Preferred arylalkenyls are those wherein aryl is phenyl and the alkenyl consists of about 3 to about 6 atoms. The arylalkenyl can be optionally substituted by one or more substituents. The bond to the parent moiety is through a non-aromatic carbon atom.

“Arylalkynyl” means a group derived from a aryl and alkenyl as defined herein. Preferred arylalkynyls are those wherein aryl is phenyl and the alkynyl consists of about 3 to about 6 atoms. The arylalkynyl can be optionally substituted by one or more substituents. The bond to the parent moiety is through a non-aromatic carbon atom.

The suffix “ene” on alkyl, aryl, heterocycloalkyl, etc. indicates a divalent moiety, e.g., —CH₂CH₂— is ethylene, and 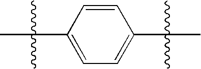
is para-phenylene.

The term “optionally substituted” means optional substitution with the specified groups, radicals or moieties, in available position or positions.

Substitution on a cycloalkylalkyl, heterocycloalkylalkyl, arylalkyl, or heteroarylalkyl moiety includes substitution on the ring portion and/or on the alkyl portion of the group.

When a variable appears more than once in a group, or a variable appears more than once in the structure of a formula, the variables can be the same or different.

With reference to the number of moieties (e.g., substituents, groups or rings) in a compound, unless otherwise defined, the phrases “one or more” and “at least one” mean that there can be as many moieties as chemically permitted, and the determination of the maximum number of such moieties is well within the knowledge of those skilled in the art. With respect to the compositions and methods comprising the use of the phrase “at least one” in a phrase such as “at least one cholesterol absorption inhibitor” or “at least one microsomal triglyceride transfer protein inhibitor” means one to three cholesterol absorption inhibitors and independently one to three microsomal triglyceride protein inhibitors can be administered at the same time, with preference to one of each.

As used herein, the term “composition” is intended to encompass a product comprising the specified ingredients in the specified amounts, as well as any product which results, directly or indirectly, from combination of the specified ingredients in the specified amounts.

The wavy line [symbol] as a bond generally indicates a mixture of, or either of, the possible isomers, e.g., containing (R)- and (S)-stereochemistry. For example, 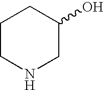
means containing both

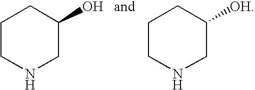

Lines drawn into the ring systems, such as, for example: 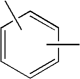
indicate that the indicated line (bond) may be attached to any of the substitutable ring carbon atoms.

It is noted that the carbon atoms for formula I may be replaced with 1 to 3 silicon atoms so long as all valency requirements are satisfied.

It should also be noted that any heteroatom with unsatisfied valences in the text or structural formulae herein is assumed to have the hydrogen atom or atoms to satisfy the valences.

Those skilled in the art will recognize that certain compounds in the structural formulae disclosed herein are tautomeric and all such tautomeric forms are contemplated herein as pan of the present invention.

As used herein, the term “cholesterol absorption inhibitor” means any agent capable of capable of inhibiting the absorption of one or more sterols, including but not limited to cholesterol, phytosterols (such as sitosterol, campesterol, stigmasterol and avenosterol), 5α-stanols (such as cholestanol, 5α-campestanol, 5α-sitostanol), and/or mixtures thereof, when administered in a therapeutically effective (sterol and/or 5α-stand absorption inhibiting) amount to a mammal or human. Non-limiting examples of cholesterol absorption inhibitors include, for example, sterol absorption inhibitors, or 5-α-stanol absorption inhibitors. “Lipid lowering agents” lower the cholesterol level in a mammal, such as a human, by not interfering with the absorption of one or more sterols in the mammal. Non-limiting examples of compounds that act as lipid lowering agents include HMG-CoA reductase inhibitors, nicotinic acid and/or nicotinic acid receptor agonists, agonists or activators of peroxisome proliferators-activated receptors (PPAR) etc. “Microsomal triglyceride transfer protein inhibitors” are any agent that is capable of inhibiting MTP.

The terms “combination therapy” or “therapeutic combination” means the administration of two or more therapeutic agents, such as a sterol absorption inhibitor and a MTP to manage, for example, the lipid levels of a mammal, to treat, prevent or ameloriate athrosclerosis in a mammal or to mitigation, preventor ameliorate the symptoms or development of hepatic steatosis in a mammal. The combinations and treatments of the present invention can be administered by any suitable means which produce contact of these compounds with the site of action in the body, for example in the plasma, liver or small intestine of a subject (mammal or human or other animal). Such administration includes coadministration of these therapeutic agents in a substantially simultaneous manner, such as in a single tablet or capsule having a fixed ratio of active ingredients or in multiple, separate capsules for each therapeutic agent. Also, such administration includes use of each type of therapeutic agent in a sequential manner. In either case, the treatment using the combination therapy will provide beneficial effects in treating the condition. A potential advantage of the combination therapy disclosed herein may be a reduction in the required amount of an individual therapeutic compound or the overall total amount of therapeutic compounds that are effective in treating the condition. By using a combination of therapeutic agents, the side effects of the individual compounds can be reduced as compared to a monotherapy, which can improve patient compliance. Also, therapeutic agents can be selected to provide a broader range of complimentary effects or complimentary modes of action.

As discussed above, the therapeutic combinations and methods of the present invention may comprise one or more substituted azetidinone or substituted β-lactam sterol absorption inhibitors discussed in detail below. As used herein, “sterol absorption inhibitor” means a compound capable of inhibiting the absorption of one or more sterols, including but not limited to cholesterol, phytosterols (such as sitosterol, campesterol, stigmasterol and avenosterol), 5α-stanols (such as cholestanol, 5α-campestanol, 5α-sitostanol), and/or mixtures thereof, when administered in a therapeutically effective (sterol and/or 5α-stanol absorption inhibiting) amount to a mammal or human.

Non-limiting examples of suitable substituted azetidinones and methods of making the same include those disclosed in U.S. Pat. Nos. RE 37,721, 5,306,817, 5,561,227, 5,618,707, 5,624,920, 5,631,365, 5,656,624, 5,627,176, 5,633,246, 5,661,145, 5,688,785, 5,688,787, 5,688,990, 5,698,548, 5,728,827, 5,739,321, 5,744,467, 5,756,470, 5,767,115, 5,846,966, 5,856,473, 5,886,171, 5,919,672, 6,093,812, 6,096,883, 6,133,001, 6,207,822, 6,627,757, 6,632,933, U.S. Patent Publication Nos. 2003/0105028, 2004/0180860, 2004/0180861, and 2004/0198700, N-sulfonyl-2-azetidinones such as are disclosed in U.S. Pat. No. 4,983,597, ethyl 4-(2-oxoazetidin-4-yl)phenoxy-alkanoates such as are disclosed in Ram et al., Indian J. Chem. Sect, B. 29B, 12 (1990), p. 1134-7, and diphenyl azetidinones and derivatives disclosed in U.S. Patent Publication Nos. 2002/0039774, 2002/0128252, 2002/0128253, 2002/0137689, 2004/0082561, and PCT Published Application Nos. WO 2002/066464, WO 04/000805, WO 04/005247, WO 04/000804, WO 04/000803, WO 04/014947, WO 04/087655, WO 05/009955, WO 05/023305, WO 05/021495, WO 05/021497, WO 05/044256, WO 05/042692, WO 05/033100, WO 05/030225, WO 05/047248, WO 05/046662, WO 05/061451, WO 05/061452, WO 05/062824, WO 05/02897, WO 05/000353, as well as the acetidiones disclosed in U.S. Patent Publication Nos. 2004/0077623, 2002/0137689, 2004/0067913, each of which is incorporated by reference herein.

In one embodiment, substituted azetidinones useful in the compositions, therapeutic combinations and methods of the present invention are represented by formula (I) below: 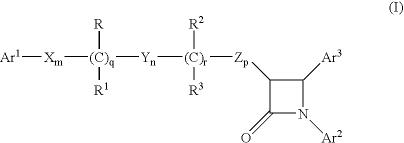
or pharmaceutically acceptable salts or solvates of the compounds of formula (I), wherein, in formula (I) above:

Ar¹ and Ar² are independently selected from the group consisting of aryl and R⁴-substituted aryl;

Ar³ is aryl or R⁵-substituted aryl;

X, Y and Z are independently selected from the group consisting of —CH₂—, —CH(lower alkyl)- and —C(dilower alkyl)-;

R and R² are independently selected from the group consisting of —OR⁶, —O(CO)R⁶, —O(CO)OR⁹ and —O(CO)NR⁶R⁷;

R¹ and R³ are independently selected from the group consisting of hydrogen, lower alkyl and aryl;

q is 0 or 1; r is 0 or 1; m, n and p are independently selected from 0, 1, 2, 3 or 4; provided that at least one of q and r is 1, and the sum of m, n, p, q and r is 1, 2, 3, 4, 5 or 6; and provided that when p is 0 and r is 1, the sum of m, q and n is 1, 2, 3, 4 or 5;

R⁴ is 1-5 substituents independently selected from the group consisting of lower alkyl, —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, (lower alkylene)COOR⁶, —CH═CH—COOR⁶, —CF₃, —CN, —NO₂ and halogen;

R⁵ is 1-5 substituents independently selected from the group consisting of —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, -(lower alkylene)COOR⁶ and —CH═CH—COOR⁶;

R⁶, R⁷ and R⁸ are independently selected from the group consisting of hydrogen, lower alkyl, aryl and aryl-substituted lower alkyl; and

R⁹ is lower alkyl, aryl or aryl-substituted lower alkyl.

Preferably, R⁴ is 1-3 independently selected substituents, and R⁵ is preferably 1-3 independently selected substituents.

Preferred compounds of formula (I) are those in which Ar¹ is phenyl or R⁴-substituted phenyl, more preferably (4-R⁴)-substituted phenyl. Ar² is preferably phenyl or R⁴-substituted phenyl, more preferably (4-R⁴)-substituted phenyl. Ar³ is preferably R⁵-substituted phenyl, more preferably (4-R⁵)-substituted phenyl. When Ar¹ is (4-R⁴)-substituted phenyl, R⁴ is preferably a halogen. When Ar² and Ar³ are R⁴- and R⁵-substituted phenyl, respectively, R⁴ is preferably halogen or —OR⁶ and R⁵ is preferably —OR⁶, wherein R⁶ is lower alkyl or hydrogen. Especially preferred are compounds wherein each of Ar¹ and Ar² is 4-fluorophenyl and Ar³ is 4-hydroxyphenyl or 4-methoxyphenyl.

X, Y and Z are each preferably —CH₂—. R¹ and R³ are each preferably hydrogen. R and R² are preferably —OR⁶ wherein R⁶ is hydrogen, or a group readily metabolizable to a hydroxyl (such as —O(CO)R⁶, —O(CO)OR⁹ and —O(CO)NR⁶R⁷, defined above).

The sum of m, n, p, q and r is preferably 2, 3 or 4, more preferably 3. Preferred are compounds wherein m, n and r are each zero, q is 1 and p is 2.

Also preferred are compounds of formula (I) in which p, q and n are each zero, r is 1 and m is 2 or 3. More preferred are compounds wherein m, n and r are each zero, q is 1, p is 2, Z is —CH₂— and R is —OR⁶, especially when R⁶ is hydrogen.

Also more preferred are compounds of formula (I) wherein p, q and n are each zero, r is 1, m is 2, X is —CH₂— and R² is —OR⁶, especially when R⁶ is hydrogen.

Another group of preferred compounds of formula (I) is that in which Ar¹ is phenyl or R⁴-substituted phenyl, Ar² is phenyl or R⁴-substituted phenyl and Ar³ is R⁵-substituted phenyl. Also preferred are compounds in which Ar¹ is phenyl or R⁴-substituted phenyl, Ar¹ is phenyl or R⁴-substituted phenyl, Ar³ is R⁵-substituted phenyl, and the sum of m, n, p, q and r is 2, 3 or 4, more preferably 3. More preferred are compounds wherein Ar¹ is phenyl or R⁴-substituted phenyl, Ar² is phenyl or R⁴-substituted phenyl, Ar³ is R⁵-substituted phenyl, and wherein m, n and r are each zero, q is 1 and p is 2, or wherein p, q and n are each zero, r is 1 and m is 2 or 3.

In a preferred embodiment, a substituted azetidinone of formula (I) useful in the compositions, therapeutic combinations and methods of the present invention is represented by formula (II) (ezetimibe) below: 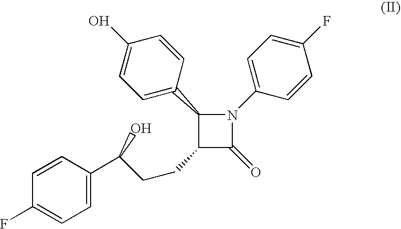
or pharmaceutically acceptable salts or solvates of the compound of formula (II). The compound of formula (II) can be in anhydrous or hydrated form. A product containing ezetimibe compound is commercially available as ZETIA® ezetimibe formulation from MSP Pharmaceuticals.

Compounds of formula I can be prepared by a variety of methods well know to those skilled in the art, for example such as are disclosed in U.S. Pat. Nos. RE 37,721, 5,631,365, 5,767,115, 5,846,966, 6,207,822, PCT Patent Application No. 02/079174, and PCT Patent Application WO 93/02048, each of which is incorporated is herein by reference.

Alternative substituted azetidinones useful in the compositions, therapeutic combinations and methods of the present invention are represented by formula (III) below: 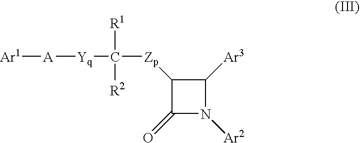
or a pharmaceutically acceptable salt thereof or a solvate thereof, wherein, in formula (III) above:

Ar¹ is R³-substituted aryl;

Ar² is R⁴-substituted aryl;

Ar³ is R⁵-substituted aryl;

Y and Z are independently selected from the group consisting of —CH₂, —CH(lower alkyl)- and —C(dilower alkyl)-;

A is selected from —O—, —S—, —S(O)— or —S(O)₂—;

R¹ is selected from the group consisting of —OR⁶, —O(CO)R⁶, —O(CO)OR⁹ and —O(CO)NR⁶R⁷; R² is selected from the group consisting of hydrogen, lower alkyl and aryl; or R¹ and R² together are ═O;

q is 1, 2 or 3;

p is 0, 1, 2, 3 or 4;

R⁵ is 1-3 substituents independently selected from the group consisting of —OR⁶, —O(CO)R⁶, —O(CO)OR⁹, —O(CH₂)₁₋₅OR⁹, —O(CO)NR⁶R⁷, —NR⁶R⁷, —NR⁶(CO)R⁷, —NR⁶(CO)OR⁹, —NR⁶(CO)NR⁷R⁸, —NR⁶SO₂-lower alkyl, —NR⁶SO₂-aryl, —CONR⁶R⁷, —COR⁶, —SO₂NR⁶R⁷, S(O)₀₋₂-alkyl, S(O)₀₋₂-aryl, —O(CH₂)₁₋₁₀—COOR⁶, —O(CH₂)₁₋₁₀CONR⁶R⁷, o-halogeno, m-halogeno, o-lower alkyl, m-lower alkyl, -(lower alkylene)-COOR⁶, and —CH═CH—COOR⁶;

R³ and R⁴ are independently 1-3 substituents independently selected from the group consisting of R⁵, hydrogen, p-lower alkyl, aryl, —NO₂, —CF₃ and p-halogeno;

R⁶, R⁷ and R⁸ are independently selected from the group consisting of hydrogen, lower alkyl, aryl and aryl-substituted lower alkyl; and R⁹ is lower alkyl, aryl or aryl-substituted lower alkyl.

Preferred compounds of formula III include those in which Ar¹ is R³-substituted phenyl, especially (4-R³)-substituted phenyl. Ar² is preferably R⁴-substituted phenyl, especially (4-R⁴)-substituted phenyl. Ar³ is preferably R⁵-substituted phenyl, especially (4-R⁵)-substituted phenyl. Mono-substitution of each of Ar¹, Ar² and Ar³ is preferred.

Y and Z are each preferably —CH₂—. R² is preferably hydrogen. R¹ is preferably —OR⁶ wherein R⁶ is hydrogen, or a group readily metabolizable to a hydroxyl (such as —O(CO)R⁶, —O(CO)OR⁹ and —O(CO)NR⁶R⁷, defined above). Also preferred are compounds wherein R¹ and R² together are ═O.

The sum of q and p is preferably 1 or 2, more preferably 1. Preferred are compounds wherein p is zero and q is 1. More preferred are compounds wherein p is zero q is 1, Y is —CH₂— and R¹ is —OR⁶, especially when R⁶ is hydrogen.

Another group of preferred compounds is that in which Ar¹ is R³-substituted phenyl, Ar² is R⁴-substituted phenyl and Ar³ is R⁵-substituted phenyl.

Also preferred are compounds wherein Ar¹ is R³-substituted phenyl, Ar² is R⁴-substituted phenyl, Ar³ is R⁵-substituted phenyl, and the sum of p and q is 1 or 2, especially 1. More preferred are compounds wherein Ar¹ is R³-substituted phenyl, Ar² is R⁴-substituted phenyl, Ar³ is R⁵-substituted phenyl, p is zero and q is 1.

A is preferably —O—.

R³ is preferably —COOR⁶, —CONR⁶R⁷, —COR⁶, —SO₂NR⁶R⁷, S(O)₀₋₂-alkyl, S(O)₀₋₂-aryl, NO₂ or halogeno. A more preferred definition for R³ is halogeno, especially fluoro or chloro.

R⁴ is preferably hydrogen, lower alkyl, —OR⁶, —O(CO)R⁶, —O(CO)OR⁹, —O(CO)NR⁶R⁷, —NR⁶R⁷, COR⁶ or halogeno, wherein R⁶ and R⁷ are preferably independently hydrogen or lower alkyl, and R⁹ is preferably lower alkyl. A more preferred definition for R⁴ is hydrogen or halogeno, especially fluoro or chloro.

R⁵ is preferably —OR⁶, —O(CO)R⁶, —O(CO)OR⁹, —O(CO)NR⁶R⁷, —NR⁶R⁷, -(lower alkylene)-COOR⁶ or —CH═CH—COOR⁶, wherein R⁶ and R⁷ are preferably independently hydrogen or lower alkyl, and R⁹ is preferably lower alkyl. A more preferred definition for R⁵ is —OR⁶, -(lower alkylene)-COOR⁶ or —CH═CH—COOR⁶, wherein R⁶ is preferably hydrogen or lower alkyl.

Methods for making compounds of Formula III are well known to those skilled in the art. Non-limiting examples of suitable methods are disclosed in U.S. Pat. No. 5,688,990, which is incorporated herein by reference.

In another embodiment, substituted azetidinones useful in the compositions, therapeutic combinations and methods of the present invention are represented by formula (IV): 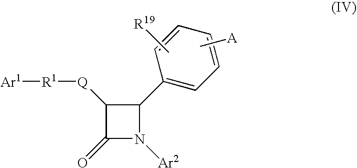
or a pharmaceutically acceptable salt thereof or a solvate thereof, wherein, in formula (IV) above:

A is selected from the group consisting of R²-substituted heterocycloalkyl, R²-substituted heteroaryl, R²-substituted benzofused heterocycloalkyl, and R²-substituted benzofused heteroaryl;

Ar¹ is aryl or R³-substituted aryl;

Ar² is aryl or R²-substituted aryl;

Q is a bond or, with the 3-position ring carbon of the azetidinone, forms the spiro group

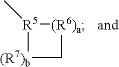

R¹ is selected from the group consisting of:
—(CH₂)_{q}—, wherein q is 2-6, provided that when Q forms a spiro ring, q can also be zero or 1;
—(CH₂)ₑ-G-(CH₂)ᵣ—, wherein G is —O—, —C(O)—, phenylene, —NR⁸— or —S(O)₀₋₂—, e is 0-5 and r is 0-5, provided that the sum of e and r is 1-6;
—(C₂-C₆ alkenylene)-; and
—(CH₂)ₜ—V—(CH₂)_{g}—, wherein V is C₃-C₆ cycloalkylene, f is 1-5 and g is 0-5, provided that the sum of f and g is 1-6;

R⁵ is selected from:

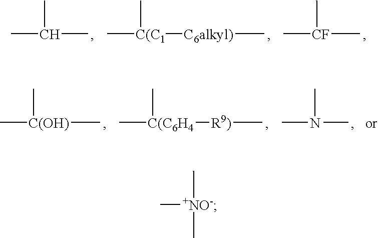

R⁶ and R⁷ are independently selected from the group consisting of —CH₂—, —CH(C₁-C₆ alkyl)-, —C(di-(C₁-C₆) alkyl), —CH═CH— and —C(C₁-C₆ alkyl)=CH—; or R⁵ together with an adjacent R⁶, or R⁵ together with an adjacent R⁷, form a —CH═CH— or a —CH═C(C₁-C₆ alkyl)- group;

a and b are independently 0, 1, 2 or 3, provided both are not zero; provided that when R⁶ is —CH═CH— or —(C₁-C₆ alkyl)=CH—, a is 1; provided that when R⁷ is —CH═CH— or —C(C₁-C₆ alkyl)=CH—, b is 1; provided that when a is 2 or 3, the R⁶'s can be the same or different; and provided that when b is 2 or 3, the R⁷'s can be the same or different;

and when Q is a bond, R¹ also can be selected from:

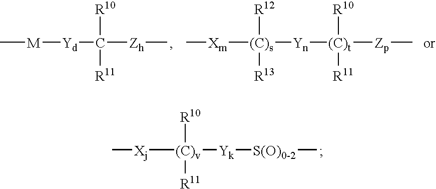

where M is —O—, —S—, —S(O)— or S(O)₂—;

X, Y and Z are independently selected from the group consisting of —CH₂—, —CH(C₁-C₆ alkyl)- and —C(di-(C₁-C₆) alkyl);

R¹⁰ and R¹² are independently selected from the group consisting of —OR¹⁴, —O(CO)R¹⁴, —O(CO)OR¹⁶ and —O(CO)NR¹⁴R¹⁵;

R¹¹ and R¹³ are independently selected from the group consisting of hydrogen, (C₁-C₆)alkyl and aryl; or R¹⁰ and R¹¹ together are ═O, or R¹² and R¹³ together are ═O;

d is 1, 2 or 3;

h is 0, 1, 2, 3 or 4;

s is 0 or 1; t is 0 or 1; m, n and p are independently 0-4; provided that at least one of s and t is 1, and the sum of m, n, p, s and t is 1-6; provided that when p is 0 and t is 1, the sum of m, s and n is 1-5; and provided that when p is 0 and s is 1, the sum of m, t and n is 1-5;

v is 0 or 1;

j and k are independently 1-5, provided that the sum of j, k and v is 1-5;

R² is 1-3 substituents on the ring carbon atoms selected from the group consisting of hydrogen, (C₁-C₁₀)alkyl, (C₂-C₁₀)alkenyl, (C₂-C₁₀)alkynyl, (C₃-C₁₀)cycloalkyl, (C₃-C₆)cycloalkenyl, R¹⁷-substituted aryl, R¹⁷-substituted benzyl, R¹⁷-substituted benzyloxy, R¹⁷-substituted aryloxy, halogeno, —NR¹⁴R¹⁵, NR¹⁴R¹⁵(C₁-C₆ alkylene)-, NR¹⁴R¹⁵C(O)(C₁-C₆ alkylene)-, —NHC(O)R¹⁶, OH, C₁-C₆ alkoxy, —OC(O)R¹⁶, —COR¹⁴, hydroxy(C₁-C₆)alkyl, (C₁-C₆)alkoxy(C₁-C₆)alkyl, NO₂R¹⁶, —S(O)₀₋₂R¹⁶, —SO₂NR¹⁴R¹⁵ and —(C₁-C₆ alkylene)COOR¹⁴; when R² is a substituent on a heterocycloalkyl ring, R² is as defined, or is ═O or 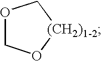
and, where R² is a substituent on a substitutable ring nitrogen, it is hydrogen, (C₁-C₆)alkyl, aryl, (C₁-C₆)alkoxy, aryloxy, (C₁-C₆)alkylcarbonyl, arylcarbonyl, hydroxy, —(CH₂)₁₋₆CONR¹⁸R¹⁸,

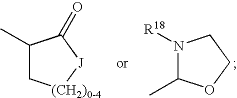

wherein J is —O— —NH—, —NR¹⁸— or —CH₂—;

R³ and R⁴ are independently selected from the group consisting of 1-3 substituents independently selected from the group consisting of (C₁-C₆)alkyl, —OR¹⁴, —O(CO)R¹⁴, —O(CO)OR¹⁶, —O(CH₂)₁₋₅OR¹⁴, —O(CO)NR¹⁴R¹⁵, —NR¹⁴R¹⁵, —NR¹⁴(CO)R¹⁵, —NR¹⁴(CO)OR¹⁶, —NR¹⁴(CO)NR¹⁵R¹⁹, —NR¹⁴SO₂R¹⁶, —COOR¹⁴, —CONR¹⁴R¹⁵, —COR¹⁴, —SO₂NR¹⁴R¹⁵, S(O)₀₋₂R¹⁶, —O(CH₂)₁₋₁₀—COOR¹⁴, —O(CH₂)₁₋₁₀CONR¹⁴R¹⁵, —(C₁-C₆ alkylene)-COOR¹⁴, —CH═CH—COOR¹⁴, —CF₃, —CN, —NO₂ and halogen;

R⁸ is hydrogen, (C₁-C₁₆)alkyl, aryl(C₁-C₆)alkyl, —C(O)R¹⁴ or —COOR¹⁴;

R⁹ and R¹⁷ are independently 1-3 groups independently selected from the group consisting of hydrogen, (C₁-C₆)alkyl, (C₁-C₆)alkoxy, —COOH, NO₂, —NR¹⁴R¹⁵, OH and halogeno;

R¹⁴ and R¹⁵ are independently selected from the group consisting of hydrogen, (C₁-C₆)alkyl, aryl and aryl-substituted (C₁-C₆)alkyl;

R¹⁶ is (C₁-C₆)alkyl, aryl or R¹⁷-substituted aryl;

R¹⁸ is hydrogen or (C₁-C₆)alkyl; and

R¹⁹ is hydrogen, hydroxy or (C₁-C₆)alkoxy.

Methods for making compounds of formula IV are well known to those skilled in the art. Non-limiting examples of suitable methods are disclosed in U.S. Pat. No. 5,656,624, which is incorporated herein by reference.

As used in formula (IV) above, “A” is preferably an R²-substituted, 6-membered heterocycloalkyl ring containing 1 or 2 nitrogen atoms. Preferred heterocycloalkyl rings are piperidinyl, piperazinyl and morpholinyl groups. The ring “A” is preferably joined to the phenyl ring through a ring nitrogen. Preferred R² substituents are hydrogen and lower alkyl. R¹⁹ is preferably hydrogen.

Ar² is preferably phenyl or R⁴-phenyl, especially (4-R⁴)-substituted phenyl. Preferred definitions of R⁴ are lower alkoxy, especially methoxy, and halogeno, especially fluoro.

Ar¹ is preferably phenyl or R³-substituted phenyl, especially (4-R³)-substituted phenyl.

There are several preferred definitions for the —R¹-Q- combination of variables:

Q is a bond and R¹ is lower alkylene, preferably propylene;

Q is a spiro group as defined above, wherein preferably R⁶ and R⁷ are each ethylene and R⁵ is

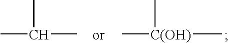

Q is a bond and R¹ is 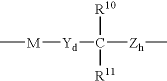
wherein the variables are chosen such that R¹ is —O—CH₂—CH(OH)—;

Q is a bond and R¹ is 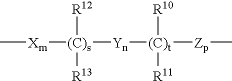
wherein the variables are chosen such that R¹ is —CH(OH)—(CH₂)₂—; and

Q is a bond and R¹ is 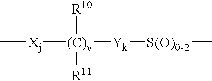
wherein the variables are chosen such that R¹ is —CH(OH)—CH₂—S(O)₀₋₂—.

In another embodiment, substituted azetidinones useful in the compositions, therapeutic combinations and methods of the present invention are represented by formula (V): 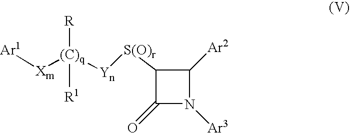
or a pharmaceutically acceptable salt thereof or a solvate thereof, wherein, in formula (V) above:

Ar¹ is aryl, R¹⁰-substituted aryl or heteroaryl;

Ar² is aryl or R⁴-substituted aryl;

Ar³ is aryl or R⁵-substituted aryl;

X and Y are independently selected from the group consisting of —CH₂—, —CH(lower alkyl)- and —C(dilower alkyl)-;

R is —OR⁶, —O(CO)R⁶, —O(CO)OR⁹ or —O(CO)NR⁶R⁷; R¹ is hydrogen, lower alkyl or aryl; or R and R¹ together are ═O;

q is 0 or 1;

r is 0, 1 or 2;

m and n are independently 0, 1, 2, 3, 4 or 5; provided that the sum of m, n and

q is 1, 2, 3, 4 or 5;

R⁴ is 1-5 substituents independently selected from the group consisting of lower alkyl, —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, -(lower alkylene)COOR⁶ and —CH═CH—COOR⁶;

R⁵ is 1-5 substituents independently selected from the group consisting of —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, —CF₃, —CN, —NO₂, halogen, -(lower alkylene)COOR⁶ and —CH═CH—COOR⁶;

R⁶, R⁷ and R⁸ are independently selected from the group consisting of hydrogen, lower alkyl, aryl and aryl-substituted lower alkyl;

R⁹ is lower alkyl, aryl or aryl-substituted lower alkyl; and

R¹⁰ is 1-5 substituents independently selected from the group consisting of lower alkyl, —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, —S(O)₀₋₂R⁹, —O(CH₂)₁₋₁₀—COOR⁶, —O(CH₂)₁₋₁₀CONR⁶R⁷, —CF₃, —CN, —NO₂ and halogen.

Within the scope of Formula V, there are included two preferred structures. In formula VA, q is zero and the remaining variables are as defined above, and in formula VB, q is 1 and the remaining variables are as defined above:

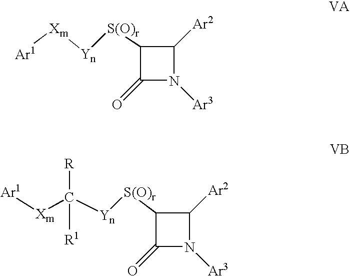

R⁴, R⁵ and R¹⁰ are each preferably 1-3 independently selected substituents as set forth above. Preferred are compounds of Formula (V) wherein Ar¹ is phenyl, R¹⁰-substituted phenyl or thienyl, especially (4-R¹⁰)-substituted phenyl or thienyl. Ar² is preferably R⁴-substituted phenyl, especially (4-R⁴)-substituted phenyl. Ar³ is preferably phenyl or R⁵-substituted phenyl, especially (4-R⁵)-substituted phenyl. When Ar¹ is R¹⁰-substituted phenyl, R¹⁰ is preferably halogeno, especially fluoro. When Ar¹ is R⁴-substituted phenyl, R⁴ is preferably —OR⁶, especially wherein R⁶ is hydrogen or lower alkyl. When Ar³ is R⁵-substituted phenyl, R⁵ is preferably halogeno, especially fluoro. Especially preferred are compounds of formula (V) wherein Ar¹ is phenyl, 4-fluorophenyl or thienyl, Ar² is 4-(alkoxy or hydroxy)phenyl, and Ar³ is phenyl or 4-fluorophenyl.

X and Y are each preferably —CH₂—. The sum of m, n and q is preferably 2, 3 or 4, more preferably 2. When q is 1, n is preferably 1 to 5.

Preferences for X, Y, Ar¹, Ar² and Ar³ are the same in each of formulae (VA) and (VB).

In compounds of formula (VA), the sum of m and n is preferably 2, 3 or 4, more preferably 2. Also preferred are compounds wherein the sum of m and n is 2, and r is 0 or 1.

In compounds of formula (VB), the sum of m and n is preferably 1, 2 or 3, more preferably 1. Especially preferred are compounds wherein m is zero and n is 1. R¹ is preferably hydrogen and R is preferably —OR⁶ wherein R⁶ is hydrogen, or a group readily metabolizable to a hydroxyl (such as —O(CO)R⁶,

—O(CO)OR⁹ and —O(CO)NR⁶R⁷, defined above), or R and R¹ together form a ═O group.

Methods for making compounds of formula V are well known to those skilled in the art. Non-limiting examples of suitable methods are disclosed in U.S. Pat. No. 5,624,920, which is incorporated herein by reference.

In another embodiment, substituted azetidinones useful in the compositions, therapeutic combinations and methods of the present invention are represented by formula (VI): 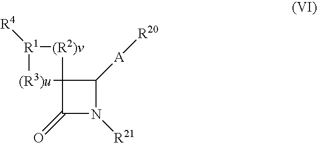
or a pharmaceutically acceptable salt thereof or a solvate thereof, wherein:

R¹ is

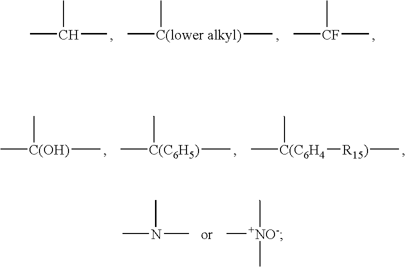

R² and R³ are independently selected from the group consisting of:

—CH₂—, —CH(lower alkyl)-, —C(di-lower alkyl)-, —CH═CH— and —C(lower alkyl)=CH—; or R¹ together with an adjacent R², or R¹ together with an adjacent R³, form a —CH═CH— or a —CH═C(lower alkyl)- group;

u and v are independently 0, 1, 2 or 3, provided both are not zero; provided that when R² is —CH═CH— or —C(lower alkyl)=CH—, v is 1; provided that when R³ is —CH═CH— or —C(lower alkyl)=CH—, u is 1; provided that when v is 2 or 3, the R²'s can be the same or different; and provided that when u is 2 or 3, the R³'s can be the same or different;

R⁴ is selected from B—(CH₂)ₘC(O)—, wherein m is 0, 1, 2, 3, 4 or 5; B—(CH₂)_{q}—, wherein q is 0, 1, 2, 3, 4, 5 or 6; B—(CH₂)ₑ-Z-(CH₂)ᵣ—, wherein Z is —O—, —C(O)—, phenylene, —N(R⁸)— or —S(O)₀₋₂—, e is 0, 1, 2, 3, 4 or 5 and r is 0, 1, 2, 3, 4 or 5, provided that the sum of e and r is 0, 1, 2, 3, 4, 5 or 6; B—(C₂-C₆ alkenylene)-; B—(C₄-C₆ alkadienylene)-; B—(CH₂)ₜ-Z-(C₂-C₆ alkenylene)-, wherein Z is as defined above, and wherein t is 0, 1, 2 or 3, provided that the sum of t and the number of carbon atoms in the alkenylene chain is 2, 3, 4, 5 or 6; B—(CH₂)_{f}—V—(CH₂)_{g}—, wherein V is C₃-C₆ cycloalkylene, f is 1, 2, 3, 4 or 5 and g is Or 1, 2, 3, 4 or 5, provided that the sum of f and g is 1, 2, 3, 4, 5 or 6; B—(CH₂)ₜ—V—(C₂-C₆ alkenylene)- or B—(C₂-C₆ alkenylene)-V—(CH₂)ₜ—, wherein V and t are as defined above, provided that the sum of t and the number of carbon atoms in the alkenylene chain is 2, 3, 4, 5 or 6; B—(CH₂)ₐ-Z-(CH₂)_{b}—V—(CH₂)_{d}—, wherein Z and V are as defined above and a, b and d are independently 0, 1, 2, 3, 4, 5 or 6, provided that the sum of a, b and d is 0, 1, 2, 3, 4, 5 or 6; or T-(CH₂)ₛ—, wherein T is cycloalkyl of 3-6 carbon atoms and s is 0, 1, 2, 3, 4, 5 or 6; or

R¹ and R⁴ together form the group

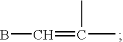

B is selected from indanyl, indenyl, naphthyl, tetrahydronaphthyl, heteroaryl or W-substituted heteroaryl, wherein heteroaryl is selected from the group consisting of pyrrolyl, pyridinyl, pyrimidinyl, pyrazinyl, triazinyl, imidazolyl, thiazolyl, pyrazolyl, thienyl, oxazolyl and furanyl, and for nitrogen-containing heteroaryls, the N-oxides thereof, or

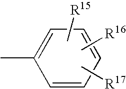

W is 1 to 3 substituents independently selected from the group consisting of lower alkyl, hydroxy lower alkyl, lower alkoxy, alkoxyalkyl, alkoxyalkoxy, alkoxycarbonylalkoxy, (lower alkoxyimino)-lower alkyl, lower alkanedioyl, lower alkyl lower alkanedioyl, allyloxy, —CF₃, —OCF₃, benzyl, R⁷-benzyl, benzyloxy, R⁷-benzyloxy, phenoxy, R⁷-phenoxy, dioxolanyl, NO₂, —N(R⁸)(R⁹), N(R⁸)(R⁹)-lower alkylene-, N(R⁸)(R⁹)-lower alkylenyloxy-, OH, halogeno, —CN, —N₃, —NHC(O)OR¹⁰, —NHC(O)R¹⁰, R¹¹O₂SNH—, (R¹¹O₂S)₂N—, —S(O)₂NH₂, —S(O)₀₋₂R⁸, tert-butyldimethyl-silyloxymethyl, —C(O)R¹², —COOR¹⁹, —CON(R⁸)(R⁹), —CH═CHC(O)R¹², -lower alkylene-C(O)R¹², R¹⁰C(O)(lower alkylenyloxy)-, N(R⁸)(R⁹)C(O)(lower alkylenyloxy)- and 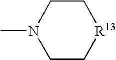
for substitution on ring carbon atoms, and the substituents on the substituted heteroaryl ring nitrogen atoms, when present, are selected from the group consisting of lower alkyl, lower alkoxy, —C(O)OR¹⁰, —C(O)R¹⁰, OH, N(R⁸)(R⁹)-lower alkylene-, N(R⁸)(R⁹)-lower alkylenyloxy-, —S(O)₂NH₂ and 2-(trimethylsilyl)-ethoxymethyl;

R⁷ is 1-3 groups independently selected from the group consisting of lower alkyl, lower alkoxy, —COOH, NO₂, —N(R⁸)(R⁹), OH, and halogeno;

R⁸ and R⁹ are independently selected from H or lower alkyl;

R¹⁰ is selected from lower alkyl, phenyl, R⁷-phenyl, benzyl or R⁷-benzyl;

R¹¹ is selected from OH, lower alkyl, phenyl, benzyl, R⁷-phenyl or R⁷-benzyl;

R¹² is selected from H, OH, alkoxy, phenoxy, benzyloxy, 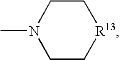
—N(R⁸)(R⁹), lower alkyl, phenyl or R⁷-phenyl;

R¹³ is selected from —O—, —CH₂—, —NH—, —N(lower alkyl)- or —NC(O)R¹⁹;

R¹⁵, R¹⁶ and R¹⁷ are independently selected from the group consisting of H and the groups defined for W; or R¹⁵ is hydrogen and R¹⁶ and R¹⁷, together with adjacent carbon atoms to which they are attached, form a dioxolanyl ring;

R¹⁹ is H, lower alkyl, phenyl or phenyl lower alkyl; and

R²⁰ and R²¹ are independently selected from the group consisting of phenyl, W-substituted phenyl, naphthyl, W-substituted naphthyl, indanyl, indenyl, tetrahydronaphthyl, benzodioxolyl, heteroaryl, W-substituted heteroaryl, benzofused heteroaryl, W-substituted benzofused heteroaryl and cyclopropyl, wherein heteroaryl is as defined above.

One group of preferred compounds of formula VI is that in which R²¹ is selected from phenyl, W-substituted phenyl, indanyl, benzofuranyl, benzodioxolyl, tetrahydronaphthyl, pyridyl, pyrazinyl, pyrimidinyl, quinolyl or cyclopropyl,

wherein W is lower alkyl, lower alkoxy, OH, halogeno, —N(R⁸)(R⁹), —NHC(O)OR¹⁰, —NHC(O)R¹⁰, NO₂, —CN, —N₃, —SH, —S(O)₀₋₂-(lower alkyl), —COOR¹⁹, —CON(R⁸)(R⁹), —COR¹², phenoxy, benzyloxy, —OCF₃—CH═C(O)R¹² or tert-butyldimethylsilyloxy, wherein R⁸, R⁹, R¹⁰, R¹² and R¹⁹ are as defined for formula IV. When W is 2 or 3 substituents, the substituents can be the same or different.

Another group of preferred compounds of formula VI is that in which R²⁰ is phenyl or W-substituted phenyl, wherein preferred meanings of W are as defined above for preferred definitions of R²¹.

More preferred are compounds of formula VI wherein R²⁰ is phenyl or W-substituted phenyl and R²¹ is phenyl, W-substituted phenyl, indanyl, benzofuranyl, benzodioxolyl, tetrahydronaphthyl, pyridyl, pyrazinyl, pyrimidinyl, quinolyl or cyclopropyl; W is lower alkyl, lower alkoxy, OH, halogeno, —N(R⁸)(R⁹), —NHC(O)OR¹⁰, —NHC(O)R¹⁰, NO₂, —CN, —N₃, —SH, —S(O)O₀₋₂-(lower alkyl), —COOR¹⁹, —CON(R⁸)(R⁹), —COR¹², phenoxy, benzyloxy, —CH═CHC(O)R₁₂, —OCF₃ or tert-butyl-dimethyl-silyloxy, wherein when W is 2 or 3 substituents, the substituents can be the same or different, and wherein R⁸, R⁹, R¹⁰, R¹² and R¹⁹ are as defined in formula VI.

Also preferred are compounds of formula VI wherein R¹ is

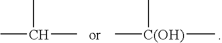

Another group of preferred compounds of formula VI is in which R² and R³ are each —CH₂— and the sum of u and v is 2, 3 or 4, with u=v=2 being more preferred.

R⁴ is preferably B—(CH₂)_{q}— or B—(CH₂)ₑ-Z-(CH₂)ᵣ—, wherein B, Z, q, e and r are as defined above. B is preferably 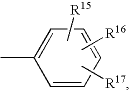
wherein R¹⁶ and R¹⁷ are each hydrogen and wherein R¹⁵ is preferably H, OH, lower alkoxy, especially methoxy, or halogeno, especially chloro.

Preferably Z is —O—, e is 0, and r is 0.

Preferably q is 0-2.

R²⁰ is preferably phenyl or W-substituted phenyl.

Preferred W substituents for R²⁰ are lower alkoxy, especially methoxy and ethoxy, OH, and —C(O)R¹², wherein R¹² is preferably lower alkoxy.

Preferably R²¹ is selected from phenyl, lower alkoxy-substituted phenyl and F-phenyl.

Especially preferred are compounds of formula VI wherein R¹ is 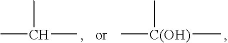
R² and R³ are each —CH₂—, u=v=2, R⁴ is B—(CH₂)_{q}—, wherein B is phenyl or phenyl substituted by lower alkoxy or chloro, q is 0-2, R²⁰ is phenyl, OH-phenyl, lower alkoxy-substituted phenyl or lower alkoxycarbonyl-substituted phenyl, and R²¹ is phenyl, lower alkoxy-substituted phenyl or F-phenyl.

An example of another useful compound of formula VI is shown below in formula VIa:

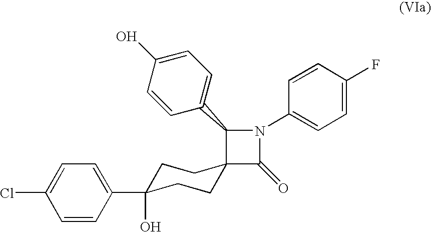

Methods for making compounds of Formula VI are well known to those skilled in the art. Non-limiting examples of suitable methods are disclosed in U.S. Pat. No. 5,698,548, which is incorporated herein by reference.

In another embodiment, substituted azetidinones useful in the compositions, therapeutic combinations and methods of the present invention are represented by Formulas (VIIA) and (VIIB): 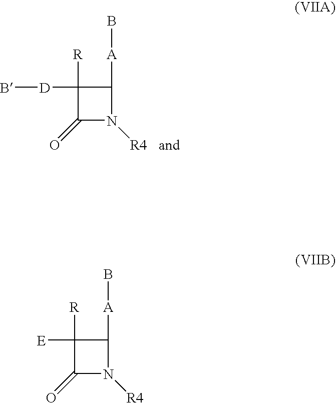
or a pharmaceutically acceptable salt or solvate thereof,
wherein:

A is —CH—OH—, —C≡C— or —(CH₂)ₚ— wherein p is 0, 1 or 2;

B is

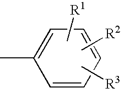

B′ is

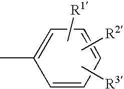

D is —(CH₂)ₘC(O)— or —(CH₂)_{q}— wherein m is 1, 2, 3 or 4 and q is 2, 3 or 4;

E is C₁₀ to C₂₀ alkyl or —C(O)—(C₉ to C₁₉)-alkyl, wherein the alkyl is straight or branched, saturated or containing one or more double bonds;

R is hydrogen, C₁-C₁₅ alkyl, straight or branched, saturated or containing one or more double bonds, or B—(CH₂)ᵣ—, wherein r is 0, 1, 2, or 3;

R¹, R², R³, R^{1′}, R^{2′}, and R^{3′} are independently selected from the group consisting of hydrogen, lower alkyl, lower alkoxy, carboxy, NO₂, NH₂, OH, halogeno, lower alkylamino, dilower alkylamino, —NHC(O)OR⁵, R⁶O₂SNH— and —S(O)₂NH₂;

R⁴ is 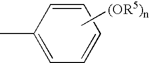
wherein n is 0, 1, 2 or 3;

R⁵ is lower alkyl; and

R⁶ is OH, lower alkyl, phenyl, benzyl or substituted phenyl wherein the substituents are 1-3 groups independently selected from the group consisting of lower alkyl, lower alkoxy, carboxy, NO₂, NH₂, OH, halogeno, lower alkylamino and dilower alkylamino; or a pharmaceutically acceptable salt thereof or a solvate thereof.

In another embodiment, sterol absorption inhibitors useful in the compositions, therapeutic combinations and methods of the present invention are represented by formula (VIII): 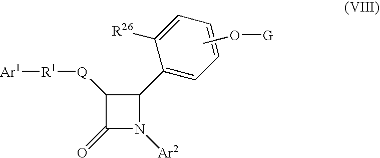
or a pharmaceutically acceptable salt thereof or a solvate thereof, wherein, in formula (VIII) above,

R²⁶ is H or OG¹;

G and G¹ are independently selected from the group consisting of 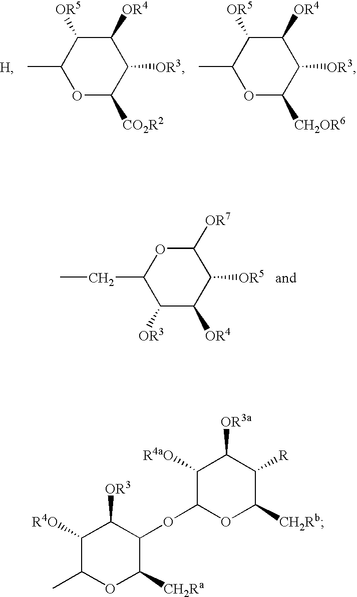
provided that when R²⁶ is H or OH, G is not H;

R, R^{a} and R^{b} are independently selected from the group consisting of H, —OH, halogeno, —NH₂, azido, (C₁-C₆)alkoxy(C₁-C₆)-alkoxy or —W—R³⁰;

W is independently selected from the group consisting of —NH—C(O)—, —O—C(O)—, —O—C(O)—N(R³¹)—, —NH—C(O)—N(R³¹)— and —O—C(S)—N(R³¹)—;

R² and R⁶ are independently selected from the group consisting of H, (C₁-C₆)alkyl, aryl and aryl(C₁-C₆)alkyl;

R³, R⁴, R⁵, R⁷, R^{3a} and R^{4a} are independently selected from the group consisting of H, (C₁-C₆)alkyl, aryl(C₁-C₆)alkyl, —C(O)(C₁-C₆)alkyl and —C(O)aryl;

R³⁰ is selected from the group consisting of R³²-substituted T, R³²-substituted-T-(C₁-C₆)alkyl, R³²-substituted-(C₂-C₄)alkenyl, R³²-substituted-(C₁-C₆)alkyl, R³²-substituted-(C₃-C₇)cycloalkyl and R³²-substituted-(C₃-C₇)cycloalkyl(C₁-C₆)alkyl;

R³¹ is selected from the group consisting of H and (C₁-C₄)alkyl;

T is selected from the group consisting of phenyl, furyl, thienyl, pyrrolyl, oxazolyl, isoxazolyl, thiazolyl, isothiazolyl, benzothiazolyl, thiadiazolyl, pyrazolyl, imidazolyl and pyridyl;

R³² is independently selected from 1-3 substituents independently selected from the group consisting of halogeno, (C₁-C₄)alkyl, —OH, phenoxy, —CF₃, —NO₂, (C₁-C₄)alkoxy, methylenedioxy, oxo, (C₁-C₄)alkylsulfanyl, (C₁-C₄)alkylsulfinyl, (C₁-C₄)alkylsulfonyl, —N(CH₃)₂, —C(O)—NH(C₁-C₄)alkyl, —C(O)—N((C₁-C₄)alkyl)₂, —C(O)—(C₁-C₄)alkyl, —C(O)—(C₁-C₄)alkoxy and pyrrolidinylcarbonyl; or R³² is a covalent bond and R³¹, the nitrogen to which it is attached and R³² form a pyrrolidinyl, piperidinyl, N-methyl-piperazinyl, indolinyl or morpholinyl group, or a (C₁-C₄)alkoxycarbonyl-substituted pyrrolidinyl, piperidinyl, N-methylpiperazinyl, indolinyl or morpholinyl group;

Ar¹ is aryl or R¹⁰-substituted aryl;

Ar² is aryl or R¹¹-substituted aryl;

Q is a bond or, with the 3-position ring carbon of the azetidinone, forms the spiro group

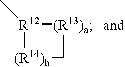

R¹ is selected from the group consisting of
—(CH₂)_{q}—, wherein q is 2-6, provided that when Q forms a spiro ring, q can also be zero or 1;
—(CH₂)ₑ-E-(CH₂)ᵣ—, wherein E is —O—, —C(O)—, phenylene, —NR²²— or —S(O)₀₋₂—, e is 0-5 and r is 0-5, provided that the sum of e and r is 1-6;
—(C₂-C₆)alkenylene-; and
—(CH₂)_{f}—V—(CH₂)_{g}—, wherein V is C₃-C₆ cycloalkylene, f is 1-5 and g is 0-5, provided that the sum of f and g is 1-6;

R¹² is

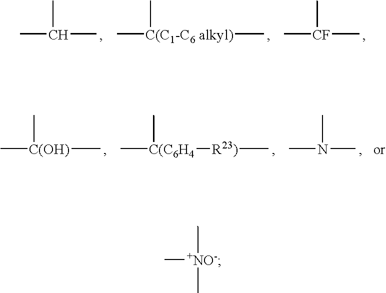

R¹³ and R¹⁴ are independently selected from the group consisting of —CH₂—, —CH(C₁-C₆ alkyl)-, —C(di-(C₁-C₆)alkyl), —CH═CH— and —C(C₁-C₆ alkyl)=CH—; or R¹² together with an adjacent R¹³, or R¹² together with an adjacent R¹⁴, form a —CH═CH— or a —CH═C(C₁-C₆ alkyl)- group;

a and b are independently 0, 1, 2 or 3, provided both are not zero;

provided that when R¹³ is —CH═CH— or —C(C₁-C₆ alkyl)=CH—, a is 1;

provided that when R¹⁴ is —CH═CH— or —C(C₁-C₆ alkyl)=CH—, b is 1;

provided that when a is 2 or 3, the R¹³'s can be the same or different; and

provided that when b is 2 or 3, the R¹⁴'s can be the same or different;

and when Q is a bond, R¹ also can be:

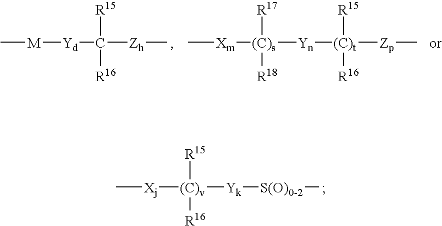

M is —O—, —S—, —S(O)— or —S(O)₂—;

X, Y and Z are independently selected from the group consisting of —CH₂—, —CH(C₁-C₆)alkyl- and —C(di-(C₁-C₆)alkyl);

R¹⁰ and R¹¹ are independently selected from the group consisting of
1-3 substituents independently selected from the group consisting of (C₁-C₆)alkyl, —OR¹⁹, —O(CO)R¹⁹, —O(CO)OR²¹, —O(CH₂)₁₋₅OR¹⁹, —O(CO)NR¹⁹R²⁰, —NR¹⁹R²⁰, —NR¹⁹(CO)R²⁰, —NR¹⁹(CO)OR²¹, —NR¹⁹(CO)NR²⁰R²⁵, —NR¹⁹SO₂R²¹, —COOR¹⁹, —CONR¹⁹R²⁰, —COR¹⁹, —SO₂NR¹⁹R²⁰, S(O)₀₋₂R²¹, —O(CH₂)₁₋₁₀—COOR¹⁹, —O(CH₂)₁₋₁₀CONR¹⁹R²⁰, —(C₁-C₆ alkylene)-COOR¹⁹, —CH═CH—COOR¹⁹, —CF₃, —CN, —NO₂ and halogen;

R¹⁵ and R¹⁷ are independently selected from the group consisting of —OR¹⁹, —O(CO)R¹⁹, —O(CO)OR²¹ and —O(CO)NR¹⁹R²⁰;

R¹⁶ and R¹⁸ are independently selected from the group consisting of H, (C₁-C₆)alkyl and aryl; or R¹⁵ and R¹⁶ together are ═O, or R¹⁷ and R¹⁸ together are ═O;

d is 1, 2 or 3;

h is 0, 1, 2, 3 or 4;

s is 0 or 1; t is 0 or 1; m, n and p are independently 0-4;

provided that at least one of s and t is 1, and the sum of m, n, p, s and t is 1-6;

provided that when p is 0 and t is 1, the sum of m, s and n is 1-5; and provided that when p is 0 and s is 1, the sum of m, t and n is 1-5;

v is 0 or 1;

j and k are independently 1-5, provided that the sum of j, k and v is 1-5;

and when Q is a bond and R¹ is 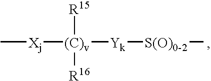
Ar¹ can also be pyridyl, isoxazolyl, furanyl, pyrrolyl, thienyl, imidazolyl, pyrazolyl, thiazolyl, pyrazinyl, pyrimidinyl or pyridazinyl;

R¹⁹ and R²⁰ are independently selected from the group consisting of H, (C₁-C₆)alkyl, aryl and aryl-substituted (C₁-C₆)alkyl;

R²¹ is (C₁-C₆)alkyl, aryl or R²⁴-substituted aryl;

R²² is H, (C₁-C₆)alkyl, aryl(C₁-C₆)alkyl, —C(O)R¹⁹ or —COOR¹⁹;

R²³ and R²⁴ are independently 1-3 groups independently selected from the group consisting of H, (C₁-C₆)alkyl, (C₁-C₆)alkoxy, —COOH, NO₂, —NR¹⁹R²⁰, —OH and halogeno; and

R²⁵ is H, —OH or (C₁-C₆)alkoxy.

Methods for making compounds of formula VIII are well known to those skilled in the art. Non-limiting examples of suitable methods are disclosed in U.S. Pat. No. 5,756,470, which is incorporated herein by reference.

In another embodiment, substituted azetidinones useful in the compositions, therapeutic combinations and methods of the present invention are represented by formula (IX) below: 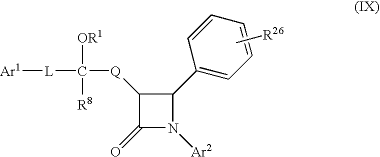
or a pharmaceutically acceptable salt or solvate thereof, wherein in Formula (IX):

R¹ is selected from the group consisting of H, G, G¹, G², —SO₃H and —PO₃H;

G is selected from the group consisting of: H,

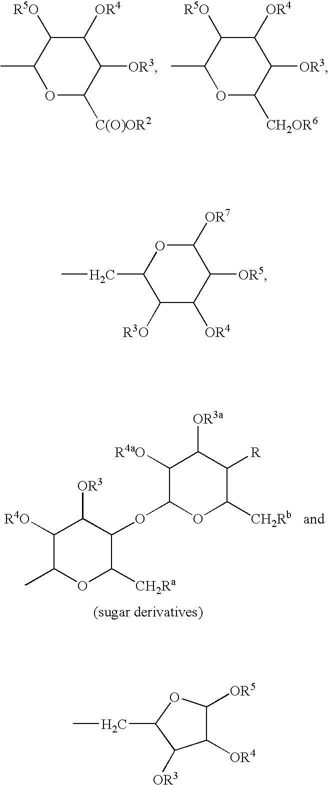

wherein R, R^{a} and R^{b} are each independently selected from the group consisting of H, —OH, halo, —NH₂, azido, (C₁-C₆)alkoxy(C₁-C₆)alkoxy or —W—R³⁰;

W is independently selected from the group consisting of —NH—C(O)—, —O—C(O)—, —O—C(O)—N(R³¹)—, —NH—C(O)—N(R³¹)— and —O—C(S)—N(R³¹)—;

R² and R⁶ are each independently selected from the group consisting of H, (C₁-C₆)alkyl, acetyl, aryl and aryl(C₁-C₆)alkyl;

R³, R⁴, R⁵, R⁷, R^{3a} and R^{4a} are each independently selected from the group consisting of H, (C₁-C₆)alkyl, acetyl, aryl(C₁-C₆)alkyl, —C(O)(C₁-C₆)alkyl and —C(O)aryl;

R³⁰ is independently selected from the group consisting of R³²-substituted T, R³²-substituted-T-(C₁-C₆)alkyl, R³²-substituted-(C₂-C₄)alkenyl, R³²-substituted-(C₁-C₆)alkyl, R³²-substituted-(C₃-C₇)cycloalkyl and R³²-substituted-(C₃-C₇)cycloalkyl(C₁-C₆)alkyl;

R³¹ is independently selected from the group consisting of H and (C₁-C₄)alkyl;

T is independently selected from the group consisting of phenyl, furyl, thienyl, pyrrolyl, oxazolyl, isoxazolyl, thiazolyl, isothiazolyl, benzothiazolyl, thiadiazolyl, pyrazolyl, imidazolyl and pyridyl;

R³² is independently selected from 1-3 substituents which are each independently selected from the group consisting of H, halo, (C₁-C₄)alkyl, —OH, phenoxy, —CF₃, —NO₂, (C₁-C₄)alkoxy, methylenedioxy, oxo, (C₁-C₄)alkylsulfanyl, (C₁-C₄)alkylsulfinyl, (C₁-C₄)alkylsulfonyl, —N(CH₃)₂, —C(O)—NH(C₁-C₄)alkyl, —C(O)—N((C₁-C₄)alkyl)₂, —C(O)—(C₁-C₄)alkyl, —C(O)—(C₁-C₄)alkoxy and pyrrolidinylcarbonyl; or R³² is a covalent bond and R³¹, the nitrogen to which it is attached and R³² form a pyrrolidinyl, piperidinyl, N-methyl-piperazinyl, indolinyl or morpholinyl group, or a (C₁-C₄)alkoxycarbonyl-substituted pyrrolidinyl, piperidinyl, N-methylpiperazinyl, indolinyl or morpholinyl group;

G¹ is represented by the structure: 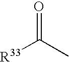
wherein R³³ is independently selected from the group consisting of unsubstituted alkyl, R³⁴-substituted alkyl, (R³⁵)(R³⁶)alkyl-,

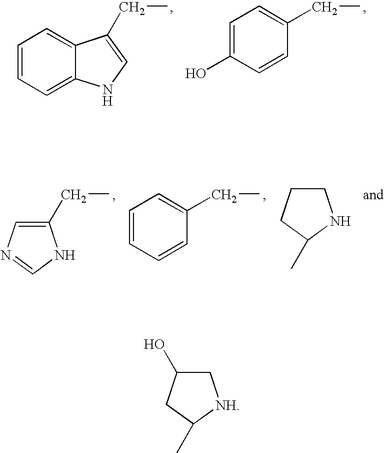

R³⁴ is one to three substituents, each R³⁴ being independently selected from the group consisting of HOOC—, HO—, HS—, (CH₃)S—, H₂N—, (NH₂)(NH)C(NH)—, (NH₂)C(O)— and HOOCCH(NH₃⁺)CH₂SS—;

R³⁵ is independently selected from the group consisting of H and NH₂—;

R³⁶ is independently selected from the group consisting of H, unsubstituted alkyl, R³⁴-substituted alkyl, unsubstituted cycloalkyl and R³⁴-substituted cycloalkyl;

G² is represented by the structure: 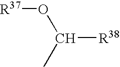
wherein R³⁷ and R³⁸ are each independently selected from the group consisting of (C₁-C₆)alkyl and aryl;

R²⁶ is one to five substituents, each R²⁶ being independently selected from the group consisting of:
a) H;
b) —OH;
c) —OCH₃;
d) fluorine;
e) chlorine;
f) —O-G;
g) —O-G¹;
h) —O-G²;
i) —SO₃H; and
j) —PO₃H;
   provided that when R¹ is H, R²⁶ is not H, —OH, —OCH₃ or —O-G;

Ar¹ is aryl, R¹⁰-substituted aryl, heteroaryl or R¹⁰-substituted heteroaryl;

Ar² is aryl, R¹¹-substituted aryl, heteroaryl or R¹¹-substituted heteroaryl;

L is selected from the group consisting of:

a) a covalent bond;

b) —(CH₂)_{q}—, wherein q is 1-6;

c) —(CH₂)ₑ-E-(CH₂)ᵣ—, wherein E is —O—, —C(O)—, phenylene, —NR²²— or —S(O)₀₋₂—, e is 0-5 and r is 0-5, provided that the sum of e and r is 1-6;

d) —(C₂-C₆)alkenylene-;

e) (CH₂)_{f}—V—(CH₂)_{g}—, wherein V is C₃-C₆cycloalkylene, f is 1-5 and g is 0-5, provided that the sum of f and g is 1-6; and

f) 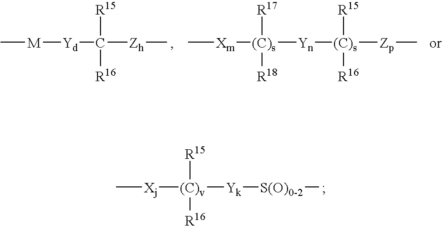
wherein M is —O—, —S—, —S(O)— or —S(O)₂—;

X, Y and Z are each independently selected from the group consisting of —CH₂—, —CH(C₁-C₆)alkyl- and —C(di-(C₁-C₆)alkyl)-;

R⁸ is selected from the group consisting of H and alkyl;

R¹⁰ and R¹¹ are each independently selected from the group consisting of 1-3 substituents which are each independently selected from the group consisting of (C₁-C₆)alkyl, —OR¹⁹, —O(CO)R¹⁹, —O(CO)OR²¹, —O(CH₂)₁₋₅R¹⁹, —O(CO)NR¹⁹R²⁰, —NR¹⁹R²⁰, —NR¹⁹(CO)R²⁰, —NR¹⁹(CO)OR²¹, —NR¹⁹(CO)NR²⁰R²⁵, —NR¹⁹SO₂R²¹, —COOR¹⁹, —CONR¹⁹R²⁰, —COR¹⁹, —SO₂NR¹⁹R²⁰, S(O)₀₋₂R²¹, —O(CH₂)₁₋₁₀—COOR¹⁹, —O(CH₂)₁₋₁₀CONR¹⁹R²⁰, —(C₁-C₆ alkylene)-COOR¹⁹, —CH═CH—COOR¹⁹, —CF₃, —CN, —NO₂ and halo;

R¹⁵ and R¹⁷ are each independently selected from the group consisting of —OR¹⁹, —OC(O)R¹⁹, —OC(O)OR²¹, —OC(O)NR¹⁹R²⁰;

R¹⁶ and R¹⁸ are each independently selected from the group consisting of H, (C₁-C₆)alkyl and aryl;

or R¹⁵ and R¹⁶ together are —O, or R¹⁷ and R¹⁸ together are ═O;

d is 1, 2 or 3;

h is 0, 1, 2, 3 or 4;

s is 0 or 1;

t is 0 or 1;

m, n and p are each independently selected from 0-4;

provided that at least one of s and t is 1, and the sum of m, n, p, s and t is 1-6; provided that when p is 0 and t is 1, the sum of m, n and p is 1-5; and provided that when p is 0 and s is 1, the sum of m, t and n is 1-5;

v is 0 or 1;

j and k are each independently 1-57 provided that the sum of j, k and v is 1-5;

Q is a bond, —(CH₂)_{q}—, wherein q is 1-6, or, with the 3-position ring carbon of the azetidinone, forms the spiro group

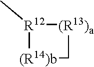

wherein R¹² is

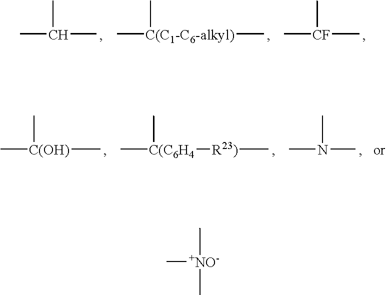

R¹³ and R¹⁴ are each independently selected from the group consisting of —CH₂—, —CH(C₁-C₆ alkyl)-, —C(di-(C₁-C₆)alkyl), —CH═CH— and —C(C₁-C₆ alkyl)=CH—; or R¹² together with an adjacent R¹³, or R¹² together with an adjacent R¹⁴, form a —CH═CH— or a —CH═C(C₁-C₆ alkyl)- group;

a and b are each independently 0, 1, 2 or 3, provided both are not zero; provided that when R¹³ is —CH═CH— or —C(C₁-C₆ alkyl)=CH—, a is 1; provided that when R¹⁴ is —CH═CH— or —C(C₁-C₆ alkyl)=CH—, b is 1; provided that when a is 2 or 3, the R¹³'s can be the same or different; and provided that when b is 2 or 3, the R¹⁴'s can be the same or different;
and when Q is a bond and L is 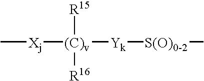
then Ar¹ can also be pyridyl, isoxazolyl, furanyl, pyrrolyl, thienyl, imidazolyl, pyrazolyl, thiazolyl, pyrazinyl, pyrimidinyl or pyridazinyl;

R¹⁹ and R²⁰ are each independently selected from the group consisting of H, (C₁-C₆)alkyl, aryl and aryl-substituted (C₁-C₆)alkyl;

R²¹ is (C₁-C₆)alkyl, aryl or R²⁴-substituted aryl;

R²² is H, (C₁-C₆)alkyl, aryl(C₁-C₆)alkyl, —C(O)R¹⁹ or —COOR¹⁹;

R²³ and R²⁴ are each independently selected from the group consisting of 1-3 substituents which are each independently selected from the group consisting of H, (C₁-C₆)alkyl, (C₁-C₆)alkoxy, —COOH, NO₂, —NR¹⁹R²⁰, —OH and halo; and

R²⁵ is H, —OH or (C₁-C₆)alkoxy.

Examples of compounds of formula (IX) which are useful in the compositions, therapeutic combinations and methods and combinations of the present invention and methods for making such compounds are disclosed in U.S. Patent Publication No. 2003/0105028 A1, filed Jun. 11, 2002, incorporated herein by reference.

An example of a useful compound of this invention is one represented by the formula X: 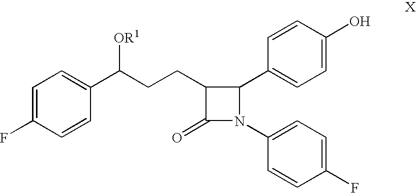
wherein R¹ is defined as above.

A more preferred compound is one represented by formula XI:

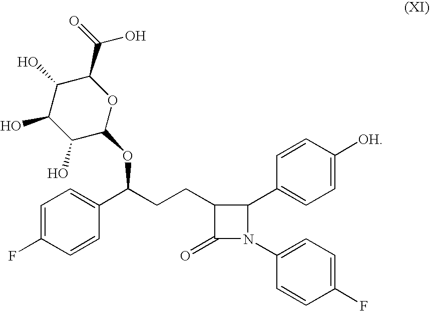

Another useful compound is represented by formula XII:

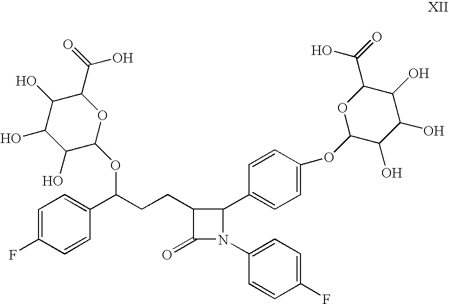

Other useful substituted azetidinone compounds include N-sulfonyl-2-azetidinones such as are disclosed in U.S. Pat. No. 4,983,597, ethyl 4-(2-oxoazetidin-4-yl)phenoxy-alkanoates such as are disclosed in Ram et al., Indian J. Chem. Sect. B. 29B, 12 (1990), p. 1134-7, and diphenyl azetidinones and derivatives disclosed in U.S. Patent Publication Nos. 2002/0039774, 2002/0128252, 2002/0128253 and 2002/0137689, and WO 2002/066464, each of which is incorporated by reference herein.

The compounds of formulae I-XII can be prepared by known methods, including the methods discussed above and, for example, WO 93/02048 describes the preparation of compounds wherein —R¹-Q- is alkylene, alkenylene or alkylene interrupted by a hetero atom, phenylene or cycloalkylene; WO 94/17038 describes the preparation of compounds wherein Q is a spirocyclic group; WO 95/08532 describes the preparation of compounds wherein —R¹-Q- is a hydroxy-substituted alkylene group; PCT/US95/03196 describes compounds wherein —R¹-Q- is a hydroxy-substituted alkylene attached to the Ar¹ moiety through an —O— or S(O)₀₋₂— group; and U.S. Pat. No. 5,633,246 describes the preparation of compounds wherein —R¹-Q- is a hydroxy-substituted alkylene group attached the azetidinone ring by a —S(O)₀₋₂— groups. Each of the aforementioned documents are incorporated by reference.

As discussed above, the compositions, therapeutic combinations and methods of the present invention comprise at least one MTP inhibitor. MTP inhibitors are well known in the art and are disclosed in, for example, US 2006/0166999 A1 and U.S. Pat. No. 6,472,414 B1, herein incorporated by reference. Other non-limiting examples of publications that disclose MTP inhibitors are as follows: WO 2005/070390, WO 2005/097131, WO 2005/046644, WO 2003/002533, WO 2001/000189, WO 2001/000184, WO2001/000183, WO 1998/050028, WO 1998/031367, WO 1998/031366, WO 1998/031225, WO 1998/003174, WO1998/003069, WO 2003/057205, WO 2001/096327, WO 2002/083654, WO 2003/0475755, WO 2005/0463644, WO 2005/087294, U.S. Pat. No. 6,256,431 B1, US2006/0089392 A1, US 2006/0058372 A1, US2005/0075367 A1, US2004/0132779 A1, US2004/0132745A1, 200410034028 A1, US 2003/0109700 A1, US 2003/0105093 A1, US 2002/0045271 A1, U.S. Pat. No. 6,878,707 B1, U.S. Pat. No. 6,369,075 B1, U.S. Pat. No. 6,235,730 B1, U.S. Pat. No. 6,197,798 B1, U.S. Pat. No. 6,472,414 B1, U.S. Pat. No. 6,281,228 B1, U.S. Pat. No. 6,066,652 B1, U.S. Pat. No. 6,066,650 B1, U.S. Pat. No. 6,066,653 B1, U.S. Pat. No. 6,057,339 B1, U.S. Pat. No. 6,034,098 B1, U.S. Pat. No. 5,990,110, U.S. Pat. No. 5,965,577, U.S. Pat. No. 5,962,440, U.S. Pat. No. 5,885,983, U.S. Pat. No. 5,883,109, U.S. Pat. No. 5,883,099, U.S. Pat. No. 5,827,875, U.S. Pat. No. 5,739,135, and U.S. Pat. No. 5,712,279. Other publications that describe MTP inhibitors includes CA 2,092,201 and EP 0643057. Each of the above-listed publications is herein incorporated by reference.

Preferred MTP inhibitors include implitapide (BAY 13-9952 or 2,2-(S)-cyclopentyl-2-{[4-(2,4-dimethyl-alpha-carbolin-9-yl)methyl]phenyl}acetic acid-(R)-phenyl-glycinol-amide), mitratapide (Janssen), which has the following structure 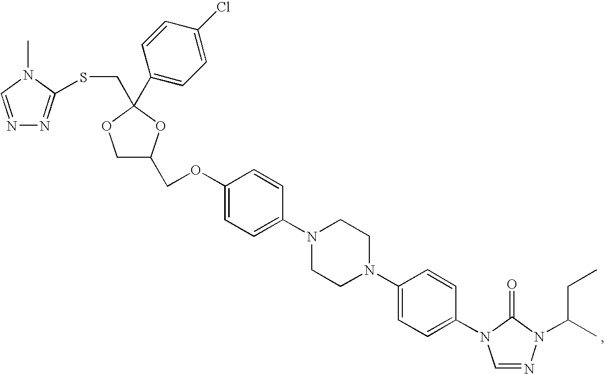
T-0126 (Tanabe), CP-3959919 (Pfizer), JTT-130 (BMC Cardiovaiscular Discord 5 30 (2005)), BMS 201038 (Bristol-Myers Squibb) and CP-346086 (Pfizer), which has the following structure

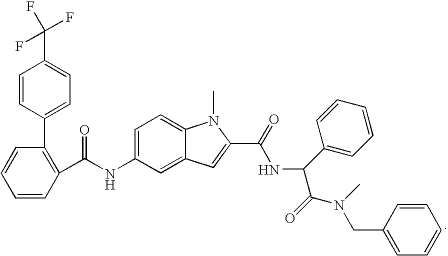

Classes of cholesterol lowering agents include the following non-limiting classes of agents: HMG-CoA reductase inhibitors; bile acid sequestrants; PPAR agonists or activators; ileal bile acid transport (“IBAT”) inhibitors (or apical sodium co-dependent bile acid transport (“ASBT”) inhibitors; nicotinic acid (niacin) and/or nicotinic acid receptor agonists; acylCoA:cholesterol O-acyhtransferase (“ACAT”) inhibitors; cholesteryl ester transfer protein (4“CETP”)⁵ inhibitors; probucol or derivatives thereof; low-density lipoprotein (“LDL”) receptor activators; omega 3 fatty acids (“3-PUFA”); natural water soluble fibers; plant sterols, plant stanols and/or fatty acid esters of plant stanols.

Non-limiting examples of suitable cholesterol biosynthesis inhibitors include competitive inhibitors of HMG-CoA reductase, the rate-limiting step in cholesterol biosynthesis, squalene synthase inhibitors, squalene epoxidase inhibitors and mixtures thereof, Non-limiting examples of suitable HMG-CoA reductase inhibitors include statins such as lovastatin (for example MEVACOR® which is available from Merck & Co.), pravastatin (for example PRAVACHOL® which is available from Bristol Meyers Squibb), fluvastatin, simvastatin (for example ZOCOR® which is available from Merck & Co.), atorvastatin, cerivastatin, CI-981, resuvastatin, rivastatin and pitavastatin (such as NK-104 of Negma Kowa of Japan), rosuvastatin; HMG-CoA reductase inhibitors, for example L-659,699 ((E,E)-11-[3′R-(hydroxy-methyl)-4′-oxo-2′R-oxetanyl]-3,5,7R-trimethyl-2,4-undecadienoic acid); squalene synthesis inhibitors, for example squalestatin 1; and squalene epoxidase inhibitors, for example, NB-598 ((E)-N-ethyl-N-(6,6-dimethyl-2-hepten-4-ynyl)-3-[(3,3′-bithiophen-5-yl)methoxy]benzene-methanamine hydrochloride) and other sterol biosynthesis inhibitors such as DMP-565. Preferred HMG-CoA reductase inhibitors include lovastatin, pravastatin and simvastatin. The most preferred HMG-CoA reductase inhibitor is simvastatin.

Generally, a total daily dosage of cholesterol biosynthesis inhibitor(s) can range from about 0.1 to about 160 mg per day, and preferably about 0.2 to about 80 mg/day in single or 2-3 divided doses.

Other cholesterol lowering agents which are contemplated by the present invention include one bile acid sequestrants. Bile acid squestrants bind bile acids in the intestine, interrupting the enterohepatic circulation of bile acids and causing an increase in the faecal excretion of steroids.

Non-limiting examples of suitable bile acid sequestrants include cholestyramine (a styrene-divinylbenzene copolymer containing quaternary ammonium cationic groups capable of binding bile acids, such as QUESTRAN® or QUESTRAN LIGHT® cholestyramine which are available from Bristol-Myers Squibb), colestipol (a copolymer of diethylenetriamine and 1-chloro-2,3-epoxypropane, such as COLESTID® tablets which are available from Pharmacia), colesevelam hydrochloride (such as WelChol® Tablets (poly(allylamine hydrochloride) cross-linked with epichlorohydrin and alkylated with 1-bromodecane and (6-bromohexyl)-trimethylammonium bromide) which are available from Sankyo), water soluble derivatives such as 3,3-ioene, N-(cycloalkyl)alkylamines and poliglusam, insoluble quaternized polystyrenes, saponins and mixtures thereof. Suitable inorganic cholesterol sequestrants include bismuth salicylate plus montmorillonite clay, aluminum hydroxide and calcium carbonate antacids.

Another embodiment of the present invention include activators or agonists of PPAR. The activators act as agonists for the peroxisome proliferator-activated receptors. Three subtypes of PPAR have been identified, and these are designated as peroxisome proliferator-activated receptor alpha (PPARα), peroxisome proliferator-activated receptor gamma (PPARγ) and peroxisome proliferator-activated receptor delta (PPARδ). It should be noted that PPARδ is also referred to in the literature as PPARβ and as NUC1, and each of these names refers to the same receptor.

PPARα regulates the metabolism of lipids. PPARα is activated by fibrates and a number of medium and long-chain fatty acids, and it is involved in stimulating β-oxidation of fatty acids. The PPARγ receptor subtypes are involved in activating the program of adipocyte differentiation and are not involved in stimulating peroxisome proliferation in the liver. PPARδ has been identified as being useful in increasing high density lipoprotein (HDL) levels in humans. See, e.g., WO 97/28149.

PPARα activator compounds are useful for, among other things, lowering triglycerides, moderately lowering LDL levels and increasing HDL levels. Useful examples of PPARα activators include fibrates.

Non-limiting examples of suitable fibric acid derivatives (“fibrates”) include clofibrate (such as ethyl 2-(p-chlorophenoxy)-2-methyl-propionate, for example ATROMID-S® Capsules which are commercially available from Wyeth-Ayerst); gemfibrozil (such as 5-(2,5-dimethylphenoxy)-2,2-dimethylpentanoic acid, for example LOPID® tablets which are commercially available from Pfizer); ciprofibrate (C.A.S. Registry No. 52214-84-3, see U.S. Pat. No. 3,948,973 which is incorporated herein by reference); bezafibrate (C.A.S. Registry No. 41859-67-0, see U.S. Pat. No. 3,781,328 which is incorporated herein by reference); clinofibrate (C.A.S. Registry No. 30299-08-2, see U.S. Pat. No. 3,716,583 which is incorporated herein by reference); binifibrate (C.A.S. Registry No. 69047-39-8, see BE 884722 which is incorporated herein by reference); lifibrol (C.A.S. Registry No. 96609-16-4); fenofibrate (such as TRICOR® micronized fenofibrate (2-[4-(4-chlorobenzoyl)phenoxy]-2-methyl-propanoic acid, 1-methylethyl ester) which is commercially available from Abbott Laboratories or LIPANTHYL® micronized fenofibrate which is commercially available from Labortoire Founier, France) and mixtures thereof. These compounds can be used in a variety of forms, including but not limited to acid form, salt form, racemates, enantiomers, zwitterions and tautomers.

Other examples of PPARα activators useful in the practice of the present invention include suitable fluorophenyl compounds as disclosed in U.S. Pat. No. 6,028,109 which is incorporated herein by reference; certain substituted phenylpropionic compounds as disclosed in WO 00/75103, which is incorporated herein by reference; and PPARα activator compounds as disclosed in WO 98/43081, which is incorporated herein by reference.

Non-limiting examples of suitable PPARγ activators include derivatives of glitazones or thiazolidinediones, such as, troglitazone; rosiglitazone (such as AVANDIA® rosiglitazone maleate (-5-[[4-[2-(methyl-2-pyridinylamino)ethoxy]phenyl]methyl]-2,4-thiazolidinedione-2-butenedioate) commercially available from Smith Kline Beecham) and pioglitazone (such as ACTOS™ pioglitazone hydrochloride (5-[[4-[2-(5-ethyl-2-pyridinyl)ethoxy]phenyl]methyl]-2,4-]thiazolidinedione monohydrochloride) commercially available from Takeda Pharmaceuticals). Other useful thiazolidinediones include ciglitazone, englitazone, darglitazone and BRL 49653 as disclosed in WO 98/05331 which is incorporated herein by reference; PPARγ activator compounds disclosed in WO 00/76488 which is incorporated herein by reference; and PPARγ activator compounds disclosed in U.S. Pat. No. 5,994,554, which is incorporated herein by reference.

Other useful PPARγ activator compounds include certain acetylphenols as disclosed in U.S. Pat. No. 5,859,051 which is incorporated herein by reference; certain quinoline phenyl compounds as disclosed in WO 99/20275 which is incorporated herein by reference; aryl compounds as disclosed by WO 99/38845 which is incorporated herein by reference; certain 1,4-disubstituted phenyl compounds as disclosed in WO 00/63161; certain aryl compounds as disclosed in WO 01/00579 which is incorporated herein by reference; benzoic acid compounds as disclosed in WO 01/12612 and WO 01/12187, which are incorporated herein by reference; and substituted 4-hydroxy-phenylalconic acid compounds as disclosed in WO 97/31907, which is incorporated herein by reference.

PPARδ compounds are useful for, among other things, lowering triglyceride levels or raising HDL levels. Non-limiting examples of PPARδ activators include suitable thiazole and oxazole derivatives, such as C.A.S. Registry No. 317318-32-4, as disclosed in WO 01/00603, which is incorporated herein by reference; certain fluoro, chloro or thio phenoxy phenylacetic acids as disclosed in WO 97/28149 which is incorporated herein by reference; suitable non-β-oxidizable fatty acid analogues as disclosed in U.S. Pat. No. 5,093,365 which is incorporated herein by reference; and PPARδ compounds as disclosed in WO 99/04815 which is incorporated herein by reference.

Moreover, compounds that have multiple functionality for activating various combinations of PPARα, PPARγ and PPARδ are also useful with the practice of the present invention. Non-limiting examples include certain substituted aryl compounds as disclosed in U.S. Pat. No. 6,248,781; WO 00/23416; WO 00/23415; WO 00/23425; WO 00/23445; WO 00/23451; and WO 00/63153, all of which are incorporated herein by reference, are described as being useful PPARα and/or PPARγ activator compounds. Other non-limiting examples of useful PPARα and/or PPARγ activator compounds include activator compounds as disclosed in WO 97/25042 which is incorporated herein by reference; activator compounds as disclosed in WO 00/63190 which is incorporated herein by reference; activator compounds as disclosed in WO 01/21181 which is incorporated herein by reference; biaryl-oxa(thia)zole compounds as disclosed in WO 01/16120 which is incorporated herein by reference; compounds as disclosed in WO 00/63196 and WO 00/63209 which are incorporated herein by reference; substituted 5-aryl-2,4-thiazolidinediones compounds as disclosed in U.S. Pat. No. 6,008,237 which is incorporated herein by reference; arylthiazolidinedione and aryloxazolidinedione compounds as disclosed in WO 00/78312 and WO 00/78313G which are incorporated herein by reference; GW2331 or (2-(4-[difluorophenyl]-1heptylureido)ethyl]phenoxy)-2-methylbutyric compounds as disclosed in WO 98/05331 which is incorporated herein by reference; aryl compounds as disclosed in U.S. Pat. No. 6,166,049 which is incorporated herein by reference; oxazole compounds as disclosed in WO 01/17994 which is incorporated herein by reference; and dithiolane compounds as disclosed in WO 01/25225 and WO 01/25226 which are incorporated herein by reference.

Other useful PPAR activator compounds include substituted benzylthiazolidine-2,4-dione compounds as disclosed in WO 01/14349, WO 01/14350 and WO/01/04351 which are incorporated herein by reference; mercaptocarboxylic compounds as disclosed in WO 00/50392 which is incorporated herein by reference; ascofuranone compounds as disclosed in WO 00/53563 which is incorporated herein by reference; carboxylic compounds as disclosed in WO 99/46232 which is incorporated herein by reference; compounds as disclosed in WO 99/12534 which is incorporated herein by reference; benzene compounds as disclosed in WO 99/15520 which is incorporated herein by reference; o-anisamide compounds as disclosed in WO 01/21578 which is incorporated herein by reference; and PPAR activator compounds as disclosed in WO 01/40192 which is incorporated herein by reference.

The peroxisome proliferator-activated receptor(s) activator(s) are administered in a therapeutically effective amount to treat the specified condition, for example in a daily dose preferably ranging from about 50 to about 3000 mg per day, and more preferably about 50 to about 2000 mg per day, given in a single dose or 2-4 divided doses. The exact dose, however, is determined by the attending clinician and is dependent on such factors as the potency of the compound administered, the age, weight, condition and response of the patient.

In an alternative embodiment, the present invention includes the use of one or more IBAT inhibitors or ASBT inhibitors. The IBAT inhibitors can inhibit bile acid transport to reduce LDL cholesterol levels. Non-limiting examples of suitable IBAT inhibitors include benzothiepines such as therapeutic compounds comprising a 2,3,4,5-tetrahydro-1-benzothiepine 1,1-dioxide structure such as are disclosed in PCT Patent Application WO 00/38727 which is incorporated herein by reference.

Generally, a total daily dosage of IBAT inhibitor(s) can range from about 0.01 to about 1000 mg/day, and preferably about 0.1 to about 50 mg/day in single or 2-4 divided doses.

In another alternative embodiment, the methods of the present invention can further comprise nicotinic acid (niacin) and/or nicotinic acid receptor (“NAR”) agonists as lipid lowering agents.

As used herein, “nicotinic acid receptor agonist” means any compound comprising that will act as an agonist to the nicotinic acid receptor. Compounds include those that have a pyridine-3-carboxylate structure or a pyrazine-2-carboxylate structure, including acid forms, salts, esters, zwitterions and tautomers, where available. Examples of nicotinic acid receptor agonists include niceritrol, nicofuranose and acipimox (5-methylpyrazine-2-carboxylic acid 4-oxide). Nicotinic acid and NAR agonists inhibit hepatic production of VLDL and its metabolite LDL and increases HDL and apo A-1 levels. An example of a suitable nicotinic acid product is NIASPAN® (niacin extended-release tablets) which are available from Kos.

Generally, a total daily dosage of nicotinic acid can range from about 500 to about 10,000 mg/day, preferably about 1000 to about 8000 mg/day, and more preferably about 3000 to about 6000 mg/day in single or divided doses. Generally, the total daily dosage of a NAR agonist can range from about 1 to about 100 mg/day/

In another alternative embodiment, the methods of the present invention can further comprise one or more ACAT inhibitors as lipid lowering agents. ACAT inhibitors reduce LDL and VLDL levels ACAT is an enzyme responsible for esterifying excess intracellular cholesterol and may reduce the synthesis of VLDL, which is a product of cholesterol esterification, and overproduction of apo B-100-containing lipoproteins.

Non-limiting examples of useful ACAT inhibitors include avasimibe ([[2,4,6-tris(1-methylethyl)phenyl]acetyl]sulfamic acid, 2,6-bis(1-methylethyl)phenyl ester, formerly known as CI-1011), HL-004, lecimibide (DuP-128) and CL-277082 (N-(2,4-difluorophenyl)-N-[[4-(2,2-dimethylpropyl)phenyl]methyl]-N-heptylurea). See P. Chang et al., “Current, New and Future Treatments in Dyslipidaemia and Atherosclerosis”, Drugs 2000 July; 60(1); 55-93, which is incorporated by reference herein.

Generally, a total daily dosage of ACAT inhibitor(s) can range from about 0.1 to about 1000 mg/day in single or 2-4 divided doses.

In another alternative embodiment, the compositions used and methods of the present invention can further comprise one or more Cholesteryl Ester Transfer Protein (“CETP”) Inhibitors coadministered with or in combination with the compound(s) of Formulae I-X discussed above. CETP is responsible for the exchange or transfer of cholesteryl ester carrying HDL and triglycerides in VLDL.

Non-limiting examples of suitable CETP inhibitors are disclosed in PCT Patent Application No. WO 00/38721 and U.S. Pat. No. 6,147,090, which are incorporated herein by reference Pancreatic cholesteryl ester hydrolase (pCEH) inhibitors such as WAY-121898 also can be coadministered with or in combination with the fibric acid derivative(s) and sterol absorption inhibitor(s) discussed above.

Generally, a total daily dosage of CETP inhibitor(s) can range from about 0.01 to about 1000 mg/day, and preferably about 0.5 to about 20 mg/kg body weight/day in single or divided doses.

In another alternative embodiment, the methods of the present invention can further comprise probucol or derivatives thereof (such as AGI-1067 and other derivatives disclosed in U.S. Pat. Nos. 6,121,319 and 6,147,250), which can reduce LDL and HDL levels, as cholesterol lowering agents.

Generally, a total daily dosage of probucol or derivatives thereof can range from about 10 to about 2000 mg/day, and preferably about 500 to about 1500 mg/day in single or 2-4 divided doses.

In another alternative embodiment, the methods of the present invention can further comprise one or more low-density lipoprotein (LDL) receptor activators, as lipid lowering agents. Non-limiting examples of suitable LDL-receptor activators include HOE-402, an imidazolidinyl-pyrimidine derivative that directly stimulates LDL receptor activity. See M. Huettinger et al., “Hypolipidemic activity of HOE-402 is Mediated by Stimulation of the LDL Receptor Pathway”, Arterioscler. Thromb. 1993; 13:1005-12.

Generally, a total daily dosage of LDL receptor activator(s) can range from about 1 to about 1000 mg/day in single or 2-4 divided doses.

In another alternative embodiment, the methods of the present invention can further comprise fish oil, which contains Omega 3 fatty acids (3-PUFA), which can reduce VLDL and triglyceride levels, as a lipid lowering agent. Generally, a total daily dosage of fish oil or Omega 3 fatty acids can range from about 1 to about 30 grams per day in single or 2-4 divided doses.

In another alternative embodiment, the methods of the present invention can further comprise natural water soluble fibers, such as psyllium, guar, oat and pectin, which can reduce cholesterol levels. Generally, a total daily dosage of natural water soluble fibers can range from about 0.1 to about 10 grams per day in single or 2-4 divided doses.

In another alternative embodiment, methods of the present invention can further comprise plant sterols, plant stanols and/or fatty acid esters of plant stanols, such as sitostanol ester used in BENECOL® margarine, which can reduce cholesterol levels. Generally, a total daily dosage of plant sterols, plant stanols and/or fatty acid esters of plant stanols can range from about 0.5 to about 20 grams per day in single or 2-4 divided doses.

The inventive combinations may also contain an H₃ receptor antagonist/inverse agonist. Non-limiting H₃ receptor antagonists/inverse agonists are disclosed in U.S. Provisional Application Ser. Nos. 60/692,110 and 60/692,175, both filed on Jun. 20, 2005, U.S. 2002/183309, 2004/0097483, 2002/177589, 2002/111340, 2004/0122033, 2003/0186963, 2003/0130253, 2004/0248938, 2002/0058659, 2003/0135056, 2003/134835, 2003/153548, 2004/0019099, 2004/0097483, 2004/0048843, 2004/087573, 2004/092521, 2004/214856, 2004/248899, 2004/224953, 2004/224952, 2005/222151, 2005/222129, 2005/182045, 2005/171181, U.S. Pat. Nos. 6,620,839, 6,515,013, 6,559,140, 6,316,475, 6,166,060, 6,448,282, 6,008,240, 5,652,258, 6,417,218, 6,673,829, 6,756,384, 6,437,147, 6,720,328, 5,869,479, 6,849,621, 6,908,929, 6,908,926, 6,906,060, 6,884,809, 6,884,803, 6,878,736, 6,638,967, 6,610,721, 6,528,522, 6,518,287, 6,506,756, 6,489,337, 6,436,939, 6,448,282, 6,407,132, 6,355,665, 6,248,765, 6,133,291, 6,103,735, 6,080,871, 5,932,596, 5,929,089, 5,837,718, 5,821,259, 5,807,872, 5,639,775, 5,708,171, 5,578,616, 5,990,147, 6,906,081, 6,720,328 WO 95/14007, WO 99/24405 (each of which is herein incorporated by reference). Other non-limiting examples of H₃ receptor antagonists/inverse agonists are disclosed in U.S. Provisional Application Ser. No. 60/752,636 (Attorney Docket No. CV06410L01US, entitled “Phenoxypiperidines and Analogues Thereof Useful as Histamine H₃ Antagonists”, and U.S. Provisional Ser. No. 60/752,637 (Attorney Docket No. CV06411L01US), entitled “Substituted Aniline Derivatives Useful as Histamine H₃ Antagonists”, both filed on the same date as this application. Especially preferred H₃ antagonists/inverse agonists includes compounds selected from the group consisting of:

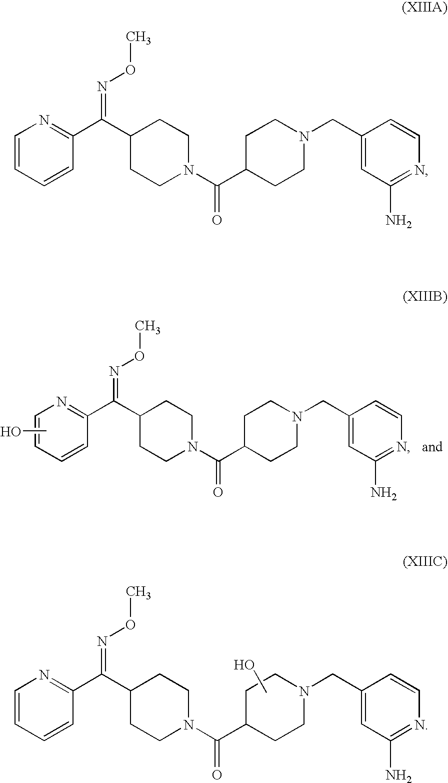

The compositions, therapeutic combinations or methods of the present invention can further comprise one or more obesity control medications. Useful obesity control medications include, but are not limited to, drugs that reduce energy intake or suppress appetite, drugs that increase energy expenditure and nutrient-partitioning agents. Suitable obesity control medications include, but are not limited to, noradrenergic agents (such as diethylpropion, mazindol, phenylpropanolamine, phentermine, phendimetrazine, phendamine tartrate, methamphetamine, phendimetrazine and tartrate); CB1 receptor antagonists (such as rimonabant); topiramate; serotonergic agents (such as sibutramine, fenfluramine, dexfenfluramine, fluoxetine, fluvoxamine and paroxtine); thermogenic agents (such as ephedrine, caffeine, theophylline, and selective β3-adrenergic agonists); an alpha-blocking agent; a kainite or AMPA receptor antagonist; a leptin-lipolysis stimulated receptor; a phosphodiesterase enzyme inhibitor; a compound having nucleotide sequences of the mahogany gene; a fibroblast growth factor-10 polypeptide; a monoamine oxidase inhibitor (such as befloxatone, moclobemide, brofaromine, phenoxathine, esuprone, befol, toloxatone, pirlindol, amiflamine, sercloremine, bazinaprine, lazabemide, milacemide and caroxazone); a compound for increasing lipid metabolism (such as evodiamine compounds); and a lipase inhibitor (such as orlistat).

Preferred pharmaceutical combinations that may be used in the methods according to the present invention include combinations comprising at least one cholesterol lowering agent, such as a sternol or 5-α-stanol according to formulae I-XII, optionally an HMG-CoA reductase inhibitor, and at least one MTP inhibitor. Especially preferred combinations include ezetimibe, optionally simvastatin and BMS 201038 and implitapide

Generally, a total dosage of the above-described obesity control medications can range from 1 to 3,000 mg/day, desirably from about 1 to 1,000 mg/day and more desirably from about 1 to 200 mg/day in single or 2-4 divided doses.

Another embodiment of the present invention is therapeutic combinations comprising a cholesterol absorption inhibitor, a MTP inhibitor and a cholesterol lowering agent. Preferred combinations include cholesterol absorption inhibitors, such as those described in formulae I to XII, and an HMG-CoA reductase inhibitor, PPAR activators, nicotinic acid (niacin) and/or nicotinic acid receptor agonists, or a bile acid sequestrant. Preferred HMG-CoA reductase inhibitors include lovastatin, pravastatin, fluvastatin, simvastatin atorvastatin, cerivastatin, CI-981>pitavastatin and rosuvastatin. Other preferred cholesterol lowering agents to be used with a cholesterol absorption inhibitor, such as those described in formulae I-XII, include cholestryamine, cholestipol, clofibrate, gemfibrozil, and fenofibrate. Especially preferred therapeutic combination is VYTORIN, which is a combination of ezetimibe and simvastatin (see U.S. Pat. No. 5,846,946, herein incorporated by reference), together with a MTP inhibitor.

Another embodiment of the present invention contemplates kits and method of treatment as described above which comprise: (a) at least one absorption agent, such as a sterol or 5-α-stanol absorption inhibitor; and (b) at least one MTP inhibitor. Suitable cholesterol absorption inhibitors include any of the compounds discussed above in formulae I-XII and suitable MTP inhibitor. A kit is contemplated when at least two separate units are combined: a pharmaceutical composition comprising at least one cholesterol absorption inhibitor and a separate pharmaceutical composition comprising at least one MTP inhibitor. The kit will preferably include directions for the administration of the separate components. The kit form is particularly advantageous when the separate components must be administered in different dosage forms (e.g., oral and parenteral) or are administered at different dosage intervals.

Prodrugs and solvates of the compounds of the invention are also contemplated herein. The term “prodrug”, as employed herein, denotes a compound that is a drug precursor which, upon administration to a subject, undergoes chemical conversion by metabolic or chemical processes to yield a compound of formula I or a salt and/or solvate thereof. A discussion of prodrugs is provided in T. Higuchi and V. Stella, Pro-drugs as Novel Delivery Systems (1987) Volume 14 of the A.C.S. Symposium Series, and in Bioreversible Carriers in Drug Design, (1987) Edward B. Roche, ed., American Pharmaceutical Association and Pergamon Press, both of which are incorporated herein by reference thereto.

For example, if a compound of formulae I-XII or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxytethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl, and the like.

Similarly, if a compound of formulae I-XII contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a compound of formulae I-XII incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇)cycloalkyl, benzyl, or R-carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C₄)alkyl and Y³ is (C₁-C₆)alkyl, carboxy(C₁-C₆)alkyl, amino(C₁-C₄)alkyl or mono-N— or di-N,N—(C₁-C₆)alkylaminoalkyl, —C(Y⁴)Y⁵ wherein Y⁴ is H or methyl and Y⁵ is mono-N— or di-N,N—(C₁-C₆)alkylamino morpholino, piperidin-1-yl or pyrrolidin-1-yl, and the like.

The compounds of formulae I-XII may exists in unsolvated as well as solvated forms, “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

“Effective amount” or “therapeutically effective amount” is meant to describe an amount of compound or a composition of the present invention effective in treating the disease state being treated and thus producing the desired therapeutic effect in a suitable patient.

The compounds of formulae I-XII form salts which are also within the scope of this invention. Reference to a compound of formulae I-XII herein is understood to include reference to salts thereof, unless otherwise indicated. The term “salt(s)”, as to employed herein, denotes acidic salts formed with inorganic and/or organic acids, as well as basic salts formed with inorganic and/or organic bases. In addition, when a compound of formulae I-XII contains both a basic moiety, such as, but not limited to a pyridine or imidazole, and an acidic moiety, such as, but not limited to a carboxylic acid, zwitterions (“inner salts”) may be formed and are included within the term is “salt(s)” as used herein. Pharmaceutically acceptable (i.e., non-toxic, physiologically acceptable) salts are preferred, although other salts are also useful Salts of the compounds of the formulae I-XII may be formed, for example, by reacting a compound of formulae I-XII with an amount of acid or base, such as an equivalent amount, in a medium such as one in which the salt precipitates or in an aqueous medium followed by lyophilization. Acids (and bases) which are generally considered suitable for the formation of pharmaceutically useful salts from basic (or acidic) pharmaceutical compounds are discussed, for example, by S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; in The Orange Book (Food & Drug Administration, Washington, D.C. on their website); and P. Heinrich Stahl, Camille G. Wermuth (Eds.), Handbook of Pharmaceutical Salts: Properties, Selection, and Use, (2002) Int'l. Union of Pure and Applied Chemistry, pp. 330-331. These disclosures are incorporated herein by reference thereto.

Exemplary acid addition salts include acetates, adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, 2-hydroxyethanesulfonates, lactates, maleates, methanesulfonates, methyl sulfates, 2-naphthalenesulfonates, nicotinates, nitrates, oxalates, pamoates, pectinates, persulfates, 3-phenylpropionates, phosphates, picrates, pivalates, propionates, salicylates, succinates, sulfates, sulfonates (such as those mentioned herein), tartarates, thiocyanates, toluenesulfonates (also known as tosylates,) undecanoates, and the like.

Exemplary basic sails include ammonium salts, alkali metal salts such as sodium, lithium, and potassium salts, alkaline earth metal salts such as calcium and magnesium salts, aluminum salts, zinc salts, salts with organic bases (for example, organic amines) such as benzathines, diethylamine, dicyclohexylamines, hydrabamines (formed with N,N-bis(dehydroabietyl)ethylenediamine), N-methyl-D-glucamines, N-methyl-D-glucamides, t-butyl amines, piperazine, phenylcyclohexylamine, choline, tromethamine, and salts with amino acids such as arginine, lysine and the like. Basic nitrogen-containing groups may be quarternized with agents such as lower alkyl halides (e.g. methyl, ethyl, propyl, and butyl chlorides, bromides and iodides), dialkyl sulfates (e.g. dimethyl, diethyl, dibutyl, and diamyl sulfates), long chain halides (e.g. decyl, lauryl, myristyl and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

All stereoisomers (for example, geometric isomers, optical isomers and the like) of the present compounds (including those of the salts, solvates and prodrugs of the compounds as well as the salts and solvates of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention. For example, if a compound formulae I-XII incorporates a double bond or a fused ring, both the cis- and trans-forms, as well as mixtures, are embraced within the scope of the invention. Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations The use of the terms “salt”, “solvate” “prodrug” and the like, is intended to equally apply to the salt, solvate and prodrug of enantiomers, stereoisomers, rotamers, tautomers, racemates or prodrugs of the inventive compounds.

Diasteromeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diasteromeric mixture by reaction with an appropriate optically active compound (e.g., chiral auxiliary such as a chiral alcohol or Moshers acid chloride), separating the diastereomers and converting (e.g., hydrolyzing) the individual diastereomers to the corresponding pure enantiomers. Also, some of the compounds of formulae I-XVII may be atropisomers (e.g., substituted biaryls) and are considered as part of this invention. Enantiomers can also be separated by use of chiral HPLC column.

Polymorphic forms of the compounds of formulae I-XII and of the salts, solvates and prodrugs of the compounds of formulae I-XII, are intended to be included in the present invention

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labelled compounds of formulae I-XVII (e.g., those labeled with ³H and ¹⁴C) are useful in compound and/or substrate tissue distribution assays. Tritiated (i.e. m ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled compounds of Formulae I-XII can generally be prepared by following procedures analogous to those disclosed in the art, by substituting an appropriate isotopically labelled reagent for a non-isotopically labelled reagent.

It should be noted that throughout the specification and Claims appended hereto any formula, compound, moiety or chemical illustration with unsatisfied valences is assumed to have the hydrogen atom to satisfy the valences unless the context indicates a bond.

The term “therapeutically effective amount” means that amount of therapeutic agents of the invention, such as the substituted azetidinone(s), the MTP inhibitor and other pharmacological or therapeutic agents which may be present that will elicit a biological or medical response of a subject, tissue, system, animal or mammal that is being sought by the administrator (such as a researcher, doctor or veterinarian) which includes alleviation of the symptoms, prevention, slowing or halting of progression of one or more conditions associated with lipid management, athrosclerosis or hepatic steatosis.

The daily dose of the compound of formulae I-XII administered to the mammal can range from about 1 to about 1000 mg per day, preferably about 1 to about mg/day, and more preferably about 100 mg per day, given in a single dose or 2-4 divided doses. The exact dose, however, is determined by the attending clinician and is dependent on the potency of the compound administered, the age, weight, condition and response of the patient.

Generally, the range for daily dose of a MTP inhibitor could be determined by one skilled in the art based upon publications incorporated herein by reference. However, the exact does is determined by the attending clinician and is dependent upon the potency of the compound administered, the age, weight, condition and response of the patient.

For administration of pharmaceutically acceptable salts of the above compounds, the weights indicated above refer to the weight of the acid equivalent or the base equivalent of the therapeutic compound derived from the salt.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 0.1 to about 7.5 percent active ingredient, Suitable solid carriers are known in the art, e.g. magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18th Edition, (1990), Mack Publishing Co., Easton, Pa.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions, suspensions and solids in powder form, which may be in combination with a pharmaceutically acceptable carrier, such as a compressed gas, egg. HFA.

Also included are solid form preparations which are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

Preferably the compound is administered orally.

Preferably, the pharmaceutical preparation is in a unit dosage form. In such form, the preparation is subdivided into suitably sized unit doses containing appropriate quantities of the active component, e.g., an effective amount to achieve the desired purpose.

The quantity of active compound in a unit dose of preparation may be varied or adjusted from about 1 to about 500 mg, preferably from about 1 mg to about 250 mg, more preferably from about 1 mg to about 100 mg, according to the particular application.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds of the invention and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 1 mg/day to about 500 mg/day, preferably 1 mg/day to 100 mg/day, in two to four divided doses.

Some useful terms are described below:

Capsule—refers to a special container or enclosure made of methyl cellulose, polyvinyl alcohols, or denatured gelatins or starch for holding or containing compositions comprising the active ingredients. Hard shell capsules are typically made of blends of relatively high gel strength bone and pork skin gelatins. The capsule itself may contain small amounts of dyes, opaquing agents, plasticizers and preservatives.

Tablet—refers to a compressed or molded solid dosage form containing the active ingredients with suitable diluents. The tablet can be prepared by compression of mixtures or granulations obtained by wet granulation, dry granulation or dry blending.

Oral gels—refers to the active ingredients dispersed or solubilized in a hydrophillic semi-solid matrix.

Powders for constitution—refers to powder blends containing the active ingredients and suitable diluents which can be suspended or solubilized in water or juices.

Diluent—refers to substances that usually make up the major portion of the composition or dosage form. Suitable diluents include sugars such as lactose, sucrose, mannitol and sorbitol; starches derived from wheat, corn, rice and potato; and celluloses such as microcrystalline cellulose. The amount of diluent in the composition can range from about 10 to about 90% by weight of the total composition, preferably from about 25 to about 75%, more preferably from about 30 to about 60% by weight, even more preferably from about 12 to about 60%.

Disintegrants—refers to materials added to the composition to help it break apart (disintegrate) and release the medicaments. Suitable disintegrants include starches; “cold water soluble” modified starches such as sodium carboxymethyl starch; natural and synthetic gums such as locust bean, karaya, guar, tragacanth and agar; cellulose derivatives such as methylcellulose and sodium carboxymethylcellulose; microcrystalline celluloses and cross-linked microcrystalline celluloses such as sodium croscarmellose; alginates such as alginic acid and sodium to alginate; clays such as bentonites; and effervescent mixtures. The amount of disintegrant in the composition can range from about 2 to about 15% by weight of the composition, more preferably from about 4 to about 10% by weight.

Binders—refers to substances that bind or “glue” powders together and make them cohesive by forming granules, thus serving as the “adhesive” in the formulation. Binders add cohesive strength already available in the diluent or bulking agent. Suitable binders include sugars such as sucrose; starches derived from wheat, corn rice and potato; natural gums such as acacia, gelatin and tragacanth; derivatives of seaweed such as alginic acid, sodium alginate and ammonium calcium alginate; cellulosic materials such as methylcellulose and sodium carboxymethylcellulose and hydroxypropylmethylcellulose; polyvinylpyrrolidone; and inorganics such as magnesium aluminum silicate. The amount of binder in the composition can range from about 2 to about 20% by weight of the composition, more preferably from about 3 to about 10% by weight, even more preferably from about 3 to about 6% by weight.

Lubricant—refers to a substance added to the dosage form to enable the tablet, granules, etc. after it has been compressed, to release from the mold or die by reducing friction or wear. Suitable lubricants include metallic stearates such as magnesium stearate, calcium stearate or potassium stearate; stearic acid; high melting point waxes; and water soluble lubricants such as sodium chloride, sodium benzoate, sodium acetate, sodium oleate, polyethylene glycols and d′l-leucine. Lubricants are usually added at the very last step before compression, since they must be present on the surfaces of the granules and in between them and the parts of the tablet press. The amount of lubricant in the composition can range from about 0.2 to about 5% by weight of the composition, preferably from about 0.5 to about 2%, more preferably from about 0.3 to about 1.5% by weight.

Glidents—materials that prevent caking and improve the flow characteristics of granulations, so that flow is smooth and uniform. Suitable glidents include silicon dioxide and talc. The amount of glident in the composition can range from about 0.1% to about 5% by weight of the total composition, preferably from about 0.5 to about 2% by weight.

Coloring agents—excipients that provide coloration to the composition or the dosage form. Such excipients can include food grade dyes and food grade dyes to adsorbed onto a suitable adsorbent such as clay or aluminum oxide. The amount of the coloring agent can vary from about 0.1 to about 5% by weight of the composition, preferably from about 0.1 to about 1%.

Bioavailability—refers to the rate and extent to which the active drug ingredient or therapeutic moiety is absorbed into the systemic circulation from an administered is dosage form as compared to a standard or control.

It will be appreciated by those skilled in the art that changes could be made to the embodiments described above without departing from the broad inventive concept thereof. It is understood, therefrom that this invention is not limited to the particular embodiments disclosed, but it is intended to cover modifications that are within the spirit and scope of the invention, as defined by the appended claims.

What is claimed is:

1. A pharmaceutical combination comprising an effective amount of at least one cholesterol absorption inhibitor and at least one microsomal triglyceride transfer protein inhibitor (MTP).

2. The pharmaceutical combination according to claim 1, wherein the cholesterol absorption is a sterol or 5-α-stanol absorption inhibitor.

3. The pharmaceutical combination according to claim 2, wherein the sterol or 5-α-stanol absorption inhibitor is a compound of formula (I): 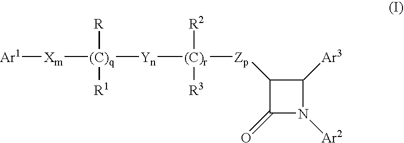
or pharmaceutically acceptable salts or solvates thereof,
wherein, in formula (I):
Ar¹ and Ar² are independently selected from the group consisting of aryl and R⁴-substituted aryl;
Ar³ is aryl or R⁵-substituted aryl;
X, Y and Z are independently selected from the group consisting of —CH₂—, —CH(lower alkyl)- and —C(dilower alkyl)-;
R and R² are independently selected from the group consisting of —OR⁶, —O(CO)R⁶, —O(CO)OR⁹ and —O(CO)NR⁶R⁷;
R¹ and R³ are independently selected from the group consisting of hydrogen, lower alkyl and aryl;
q is 0 or 1; r is 0 or 1; m, n and p are independently selected from 0, 1, 2, 3 or 4; provided that at least one of q and r is 1, and the sum of m, n, p, q and r is 1, 2, 3, 4, 5 or 6; and provided that when p is 0 and r is 1, the sum of m, q and n is 1, 2, 3, 4 or 5;
R⁴ is 1-5 substituents independently selected from the group consisting of lower alkyl, —OR⁶, —O(COOR⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, -(lower alkylene)COOR⁶, —CH—CH—COOR⁶, —CF₃—CN, —NO₂ and halogen;
R⁵ is 1-5 substituents independently selected from the group consisting of —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, -(lower alkylene)COOR⁶ and —CH═CH—COOR⁶;
R⁶, R⁷ and R⁸ are independently selected from the group consisting of hydrogen, lower alkyl, aryl and aryl-substituted lower alkyl; and
R⁹ is lower alkyl, aryl or aryl-substituted lower alkyl.

4. The pharmaceutical combination according to claim 3, wherein the sterol or 5-α-stanol absorption inhibitor is a compound of formula (II): 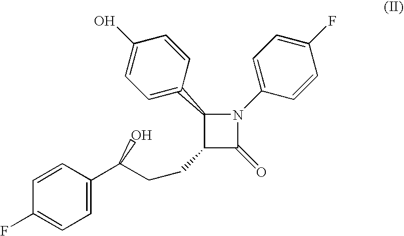
or pharmaceutically acceptable salts or solvates thereof.

5. The pharmaceutical combination according to claim 2, wherein the sterol or 5-α-stanol absorption inhibitor is a compound of formula (III): 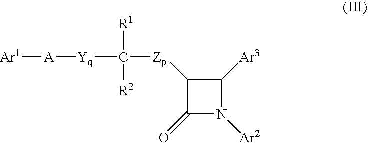
or a pharmaceutically acceptable salt thereof or a solvate thereof,
wherein, in formula (III) above:
Ar¹ is R³-substituted aryl;
Ar² is R⁴-substituted aryl;
Ar³ is R⁵-substituted aryl;
Y and Z are independently selected from the group consisting of —CH₂—, —CH(lower alkyl)- and —C(dilower alkyl)-;
A is selected from —O—, —S—, —S(O)— or —S(O)₂—,
R¹ is selected from the group consisting of —OR⁶, —O(CO)R⁶, —O(CO)OR⁹ and —O(CO)NR⁶R⁷; R² is selected from the group consisting of hydrogen, lower alkyl and aryl; or R¹ and R² together are ═O;
q is 1, 2 or 3;
p is 0, 1, 2, 3 or 4;
R⁵ is 1-3 substituents independently selected from the group consisting of —OR⁶, —O(CO)R⁶, —O(CO)OR⁹, —O(CH₂)₁₋₅OR⁹, —O(CO)NR⁶R⁷, —NR⁶R⁷, —NR⁶(CO)R⁷, —NR⁶(CO)OR⁹, —NR⁶(CO)NR⁷R⁸, —NR⁶SO₂-lower alkyl, —NR⁶SO₂-aryl, —CONR⁶R⁷, —COR⁶, —SO₂NR⁶R⁷, S(O)₀₋₂-alkyl, S(O)₀₋₂-aryl, —O(CH₂)₁₋₁₀—COOR⁶, —O(CH₂)₁₋₁₀CONR⁶R⁷, o-halogeno, m-halogeno, o-lower alkyl, m-lower alkyl, -(lower alkylene)-COOR⁶, and —CH═CH—COOR⁶;
R³ and R⁴ are independently 1-3 substituents independently selected from the group consisting of R⁵, hydrogen, p-lower alkyl, aryl, —NO₂, —CF₃ and p-halogeno;
R⁶, R⁷ and R⁸ are independently selected from the group consisting of hydrogen, lower alkyl, aryl and aryl-substituted lower alkyl; and R⁹ is lower alkyl, aryl or aryl-substituted lower alkyl.

6. The pharmaceutical combination according to claim 2, wherein the sterol or 5-α-stanol absorption inhibitor is a compound of formula (IV): 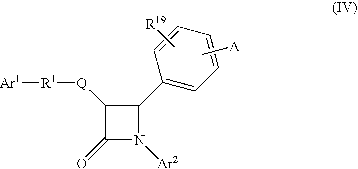
or a pharmaceutically acceptable salt thereof or a solvate thereof,
wherein, in Formula (IV):
A is selected from the group consisting of R²-substituted heterocycloalkyl, R²-substituted heteroaryl, R²-substituted benzofused heterocycloalkyl, and R²-substituted benzofused heteroaryl;
Ar¹ is aryl or R³-substituted aryl;
Ar² is aryl or R⁴-substituted aryl;
Q is a bond or, with the 3-position ring carbon of the azetidinone, forms the spiro group 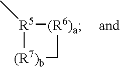
R¹ is selected from the group consisting of:
—(CH₂)_{q}—, wherein q is 2-6, provided that when Q forms a spiro ring, q can also be zero or 1;
—(CH₂)ₑ-G-(CH₂)ᵣ—, wherein G is —O—, —C(O)—, phenylene, —NR⁸— or is —S(O)₀₋₂—, e is 0-5 and r is 0-5, provided that the sum of e and r is 1-6;
—(C₂-C₆ alkenylene)-; and
—(CH₂)_{f}—V—(CH₂)_{g}—, wherein V is C₃-C₆ cycloalkylene, f is 1-5 and g is 0-5, provided that the sum of f and g is 1-6;
R⁵ is selected from: 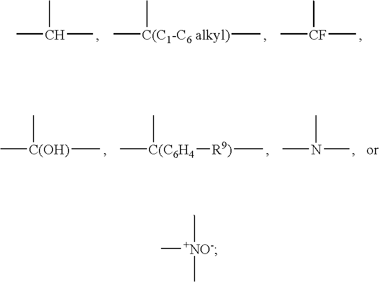
R⁶ and R⁷ are independently selected from the group consisting of —CH₂—, —CH(C₁-C₆ alkyl)-, —C(di-(C₁-C₆)alkyl), —CH═CH— and —C(C₁-C₆ alkyl)=CH—; or R⁵ together with an adjacent R⁶, or R⁵ together with an adjacent R⁷, form a —CH═CH— or a —CH═C(C₁-C₆ alkyl)- group;
a and b are independently 0, 1, 2 or 3, provided both are not zero; provided that when R⁶ is —CH═CH— or —C(C₁-C₆ alkyl)=CH—, a is 1; provided that when R⁷ is —CH═CH— or —C(C₁-C₆ alkyl)=CH—, b is 1; provided that when a is 2 or 3, the R⁶'s can be the same or different; and provided that when b is 2 or 3, the R⁷'s can be the same or different;
and when Q is a bond, R¹ also can be selected from: 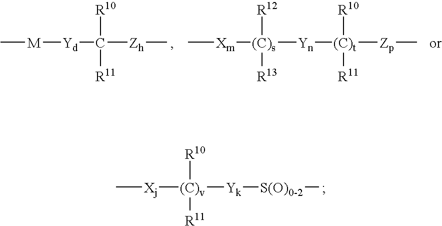
where M is —O—, —S—, —S(O)— or —S(O)₂—;
X, Y and Z are independently selected from the group consisting of —CH₂—, —CH(C₁-C₆ alkyl)- and —C(di-(C₁-C₆)alkyl);
R¹⁰ and R¹² are independently selected from the group consisting of —OR¹⁴, —O(CO)R¹⁴, —O(CO)OR¹⁶ and —O(CO)NR¹⁴R¹⁵;
R¹¹ and R¹³ are independently selected from the group consisting of hydrogen, (C₁-C₆)alkyl and aryl; or R¹⁰ and R¹¹ together are ═O, or R¹² and R¹³ together are ═O;
d is 1, 2 or 3;
h is 0, 1, 2, 3 or 4;
s is 0 or 1; t is 0 or 1; m, n and p are independently 0-4; provided that at least one of s and t is 1, and the sum of m, n, p, s and t is 1-6; provided that when p is 0 and t is 1, the sum of m, s and n is 1-5; and provided that when p is 0 and s is 1, the sum of m, t and n is 1-5;
v is 0 or 1;
j and k are independently 1-51 provided that the sum of j, k and v is 1-5;
R² is 1-3 substituents on the ring carbon atoms selected from the group consisting of hydrogen, (C₁-C₁₀)alkyl, (C₂-C₁₀)alkenyl, (C₂-C₁₀)alkynyl, (C₃-C₆)cycloalkyl, (C₃-C₆)cycloalkenyl, R¹⁷-substituted aryl, R¹⁷-substituted benzyl, R¹⁷-substituted benzyloxy, R¹⁷-substituted aryloxy, halogeno, —NR¹⁴R¹⁵, NR¹⁴R¹⁵(C₁-C₆ alkylene)-, NR¹⁴R¹⁵C(O)(C₁-C₆ alkylene)-, —NHC(O)R¹⁶, OH, C₁-C₆ alkoxy, —OC(O)R¹⁶, —COR¹⁴, hydroxy(C₁-C₆)alkyl, (C₁-C₆)alkoxy(C₁-C₆)alkyl, NO₂, —S(O)₀₋₂R¹⁶, —SO₂NR¹⁴R¹⁵ and —(C₁-C₆ alkylene)COOR¹⁴; when R² is a substituent on a heterocycloalkyl ring, R² is as defined, or is ═O or 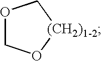
and, where R² is a substituent on a substitutable ring nitrogen, it is hydrogen, (C₁-C₆)alkyl, aryl, (C₁-C₆)alkoxy, aryloxy, (C₁-C₆)alkylcarbonyl, arylcarbonyl, hydroxy, —(CH₂)₁₋₆CONR¹⁸R¹⁸, 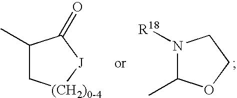
wherein J is —O—, —NH—, —NR¹⁸— or —CH₂—;
R³ and R⁴ are independently selected from the group consisting of 1-3 substituents independently selected from the group consisting of (C₁-C₆)alkyl, —OR¹⁴, —O(CO)R¹⁴, —O(CO)OR¹⁶, —O(CH₂)₁₋₅OR¹⁴, —O(CO)NR¹⁴R¹⁵, —NR¹⁴R¹⁵, —NR¹⁴(CO)R¹⁵, —NR¹⁴(CO)OR¹⁶, —NR¹⁴(CO)NR¹⁵R¹⁹, —NR¹⁴SO₂R¹⁶, —COOR¹⁴, —CONR¹⁴R¹⁵, —COR¹⁴, —SO₂NR¹⁴R¹⁵, S(O)₀₋₂R¹⁶, —O(CH₂)₁₋₁₀—COOR¹⁴, —O(CH₂)₁₋₁₀CONR¹⁴R¹⁵, —(C₁-C₆ alkylene)-COOR¹⁴, —CH═CH—COOR¹⁴, —CF₃, —CN, —NO₂ and halogen;
R⁸ is hydrogen, (C₁-C₆)alkyl, aryl(C₁-C₆)alkyl, —C(O)R¹⁴ or —COOR¹⁴;
R⁹ and R¹⁷ are independently 1-3 groups independently selected from the group consisting of hydrogen, (C₁-C₆)alkyl, (C₁-C₆)alkoxy, —COOH, NO₂, —NR¹⁴R¹⁵, OH and halogeno;
R¹⁴ and R¹⁵ are independently selected from the group consisting of hydrogen, (C₁-C₆)alkyl, aryl and aryl-substituted (C₁-C₆)alkyl;
R¹⁶ is (C₁-C₆)alkyl, aryl or R¹⁷-substituted aryl;
R¹⁸ is hydrogen or (C₁-C₆)alkyl; and
R¹⁹ is hydrogen, hydroxy or (C₁-C₆)alkoxy.

7. The pharmaceutical combination according to claim 2, wherein the sterol or 5-α-stanol absorption inhibitor is a compound of formula (V): 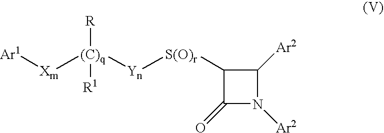
or a pharmaceutically acceptable salt thereof or a solvate thereof,
wherein, in formula (V):
Ar¹ is aryl, R¹⁰-substituted aryl or heteroaryl;
Ar² is aryl or R⁴-substituted aryl;
Ar³ is aryl or R⁵-substituted aryl;
X and Y are independently selected from the group consisting of —CH₂—, —CH(lower alkyl)- and —C(dilower alkyl)-;
R is —OR⁶, —O(CO)R⁶, —O(CO)OR⁹ or —O(CO)NR⁶R⁷; R¹ is hydrogen, lower alkyl or aryl; or R and R¹ together are ═O;
q is 0 or 1;
r is 0, 1 or 2;
m and n are independently 0, 1, 2, 3, 4 or 5; provided that the sum of m, n and q is 1, 2, 3, 4 or 5;
R⁴ is 1-5 substituents independently selected from the group consisting of lower alkyl, —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, -(lower alkylene)COOR⁶ and —CH═CH—COOR⁶;
R⁵ is 1-5 substituents independently selected from the group consisting of —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, —CF₃, —CN, —NO₂, halogen, -(lower alkylene)COOR⁶ and —CH═CH—COOR⁶;
R⁶, R⁷ and R⁸ are independently selected from the group consisting of hydrogen, lower alkyl, aryl and aryl-substituted lower alkyl;
A is lower alkyl, aryl or aryl-substituted lower alkyl; and
R¹⁰ is 1-5 substituents independently selected from the group consisting of lower alkyl, —OR⁶, —O—O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, —S(O)₀₋₂R⁹, —O(CH₂)₁₋₁₀—COOR⁶, —O(CH₂)₁₋₁₀CONR⁶R⁷, —CF₃, —CN, —NO₂ and halogen.

8. The pharmaceutical combination according to claim 2, wherein the sterol or 5-α-stanol absorption inhibitor is a compound of formula: 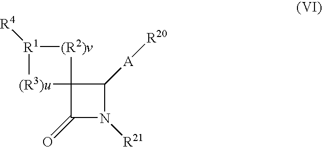
or a pharmaceutically acceptable salt thereof or a solvate thereof,
wherein, in formula (VI):
R¹ is 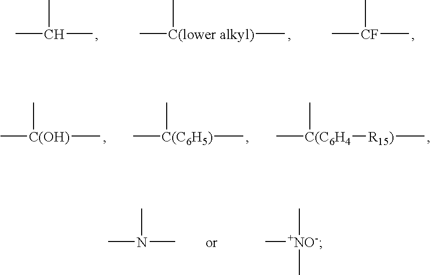
R² and R³ are independently selected from the group consisting of:
—CH₂—, —CH(lower alkyl)-, —C(di-lower alkyl)-, —CH═CH— and —C(lower alkyl)=CH—; or R¹ together with an adjacent R², or R¹ together with an adjacent R₃, form a is —CH═CH— or a —CH═C(lower alkyl)- group;
u and v are independently 0, 1, 2 or 3, provided both are not zero; provided that when R² is —CH═CH— or —C(lower alkyl)=CH—, v is 1; provided that when R³ is —CH═CH— or —C(lower alkyl)=CH—, u is 1; provided that when v is 2 or 3, the R²'s can be the same or different; and provided that when u is 2 or 3, the R³'s can be the same or different;
R⁴ is selected from B—(CH₂)ₘC(O)—, wherein m is 0, 1, 2, 3, 4 or 5; B—(CH₂)_{q}—, wherein q is 0, 1, 2, 3, 4, 5 or 6; B—(CH₂)ₑ-Z-(CH₂)ᵣ—, wherein Z is —O—, —C(O)—, phenylene, —N(R₈)— or —S(O)₀₋₂—, e is 0, 1, 2, 3, 4 or 5 and r is 0, 1, 2, 3, 4 or 5, provided that the sum of e and r is 0, 1, 2, 3, 4, 5 or 6; B—(C₂-C₆ alkenylene)-; B—(C₄-C₆ alkadienylene)-; B—(CH₂)ₜ-Z-(C₂-C₆ alkenylene)-, wherein Z is as defined above, and wherein t is 0, 1, 2 or 3, provided that the sum of t and the number of carbon atoms in the alkenylene chain is 2, 3, 4, 5 or 6; B—(CH₂)_{f}—V—(CH₂)_{g}—, wherein V is C₃-C₆ cycloalkylene, f is 1, 2, 3, 4 or 5 and g is 0, 1, 2, 3, 4 or 5, provided that the sum of f and g is 1, 2, 3, 4, 5 or 6; B—(CH₂)ₜ—V—(C₂-C₆ alkenylene)- or B—(C₂-C₆ alkenylene)-V—(CH₂)ₜ—, wherein V and t are as defined above, provided that the sum of t and the number of carbon atoms in the alkenylene chain is 2, 3, 4, 5 or 6; B—(CH₂)ₐ-Z-(CH₂)_{b}—V—(CH₂)_{d}—, wherein Z and V are as defined above and a, b and d are independently 0, 1, 2, 3, 4, 5 or 6, provided that the sum of a, b and d is 0, 1, 2, 3, 4, 5 or 6; or T-(CH₂)ₛ—, wherein T is cycloalkyl of 3-6 carbon atoms and s is 0, 1, 2, 3, 4, 5 or 6; or
R¹ and R⁴ together form the group 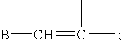
B is selected from indanyl, indenyl, naphthyl, tetrahydronaphthyl, heteroaryl or W-substituted heteroaryl, wherein heteroaryl is selected from the group consisting of pyrrolyl, pyridinyl, pyrimidinyl, pyrazinyl, triazinyl, imidazolyl, thiazolyl, pyrazolyl, thienyl, oxazolyl and furanyl, and for nitrogen-containing heteroaryls, the N-oxides thereof, or 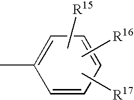
W is 1 to 3 substituents independently selected from the group consisting of lower alkyl, hydroxy lower alkyl, lower alkoxy, alkoxyalkyl, alkoxyalkoxy, alkoxycarbonylalkoxy, (lower alkoxyimino)-lower alkyl, lower alkanedioyl, lower alkyl lower alkanedioyl, allyloxy, —CF₃, —OCF₃, benzyl, R⁷-benzyl, benzyloxy, R⁷-benzyloxy, phenoxy, R⁷-phenoxy, dioxolanyl, NO₂, —N(R⁸)(R⁹), N(R⁸)(R⁹)-lower alkylene-, N(R⁸)(R⁹)-lower alkylenyloxy-, OH, halogeno, —CN, —N₃, —NHC(O)OR¹⁰, —NHC(O)R¹⁰, R¹¹O₂SNH—, (R¹¹O₂S)₂N—, —S(O)₂NH₂, —S(O)₀₋₂R⁸, tert-butyldimethyl-silyloxymethyl, —C(O)R¹², —COOR¹⁹, —CON(R⁸)(R⁹), —CH═CHC(O)R¹², -lower alkylene-C(O)R¹², R¹⁰C(O)(lower alkylenyloxy)-, N(R⁸)(R⁹)C(O)(tower alkylenyloxy)- and 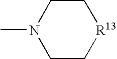
for substitution on ring carbon atoms,
and the substituents on the substituted heteroaryl ring nitrogen atoms, when present, are selected from the group consisting of lower alkyl, lower alkoxy, —C(O)OR¹⁰, —C(O)R¹⁰, OH, N(R⁸)(R⁹)-lower alkylene-, N(R⁸)(R⁹)-lower alkylenyloxy-, —S(O)₂NH₂ and 2-(trimethylsilyl)-ethoxymethyl;
R⁷ is 1-3 groups independently selected from the group consisting of lower alkyl, lower alkoxy, —COOH, NO₂, —N(R⁸)(R⁹), OH, and halogeno;
R⁸ and R⁹ are independently selected from H or lower alkyl;
R¹⁰ is selected from lower alkyl, phenyl, R⁷-phenyl, benzyl or R⁷-benzyl;
R¹¹ is selected from OH, lower alkyl, phenyl, benzyl, R⁷-phenyl or R⁷-benzyl;
R¹² is selected from H, OH, alkoxy, phenoxy, benzyloxy, 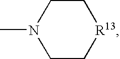
—N(R⁸)(R⁹), lower alkyl, phenyl or R₇-phenyl;
R¹³ is selected from —O—, —CH₂—, —NH—, —N(lower alkyl)- or —NC(O)R¹⁹;
R¹⁵, R¹⁶ and R¹⁷ are independently selected from the group consisting of H and the groups defined for W; or R¹⁵ is hydrogen and R¹⁶ and R¹⁷, together with adjacent carbon atoms to which they are attached, form a dioxolanyl ring;
R¹⁹ is H, lower alkyl, phenyl or phenyl lower alkyl; and
R²⁰ and R²¹ are independently selected from the group consisting of phenyl, W-substituted phenyl, naphthyl, W-substituted naphthyl, indanyl, indenyl, tetrahydronaphthyl, benzodioxolyl, heteroaryl, W-substituted heteroaryl, benzofused heteroaryl, W-substituted benzofused heteroaryl and cyclopropyl, wherein heteroaryl is as defined above.

9. The pharmaceutical combination according to claim 2, wherein the sterol or 5-α-stanol absorption inhibitor is a compound of formula by formulae (VIIA) or (VIIB): 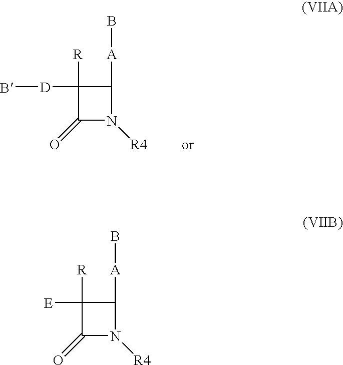
or a pharmaceutically acceptable salt or solvate thereof,
wherein in formulae (VIIA) or (VIIB):
A is —CH═CH—, —C≡C— or —(CH₂)ₚ— wherein p is 0, 1 or 2;
B is 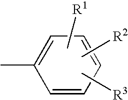
B′ is 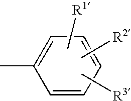
D is —(CH₂)ₘC(O)— or —(CH₂)_{q}— wherein m is 1, 2, 3 or 4 and q is 2, 3 or 4;
E is C₁₀ to C₂₀ alkyl or —C(O)—(C₉ to C₁₉)-alkyl, wherein the alkyl is straight or branched, saturated or containing one or more double bonds;
R is hydrogen, C₁-C₁₅ alkyl, straight or branched, saturated or containing one or more double bonds, or B—(CH₂)ᵣ—, wherein r is 0, 1, 2, or 3;
R¹, R², R³, R^{1′}, R^{2′}, and R^{3′} are independently selected from the group consisting of hydrogen, lower alkyl, lower alkoxy, carboxy, NO₂, NH₂, OH, halogeno, lower alkylamino, dilower alkylamino, —NHC(O)OR⁵, R⁶O₂SNH— and —S(O)₂NH₂;
R⁴ is 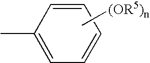
wherein n is 0, 1, 2 or 3;
R⁵ is lower alkyl; and
R⁶ is OH, lower alkyl, phenyl, benzyl or substituted phenyl wherein the substituents are 1-3 groups independently selected from the group consisting of lower alkyl, lower alkoxy, carboxy, NO₂, NH₂, OH, halogeno, lower alkylamino and dilower alkylamino.

10. The pharmaceutical combination according to claim 2, wherein the sterol or 5-α-stanol absorption inhibitor is a compound of formula (VIII): 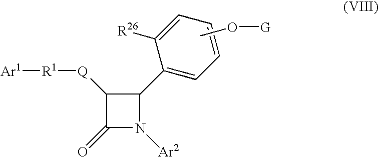
or a pharmaceutically acceptable salt thereof or a solvate thereof,
wherein, in formula (VIII) above,
R²⁶ is H or OG¹;
G and G¹ are independently selected from the group consisting of 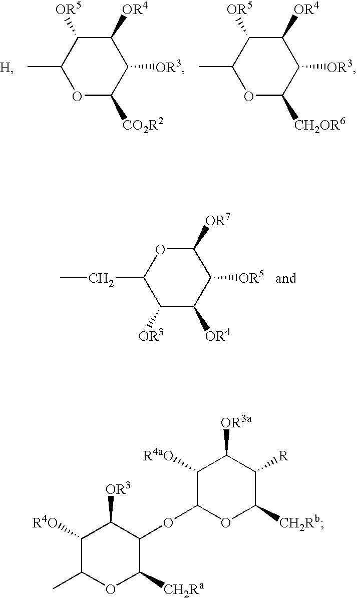
provided that when R²⁶ is H or OH, G is not H;
R, R^{a} and R^{b} are independently selected from the group consisting of H, —OH, halogeno, —NH₂, azido, (C₁-C₆)alkoxy(C₁-C₆)-alkoxy or —W—R³⁰;
W is independently selected from the group consisting of —NH—C(O)—, —O—C(O)—, —O—C(O)—N(R³¹)—, —NH—C(O)—N(R³¹)— and —O—C(S)—N(R³¹)—;
R² and R⁶ are independently selected from the group consisting of H, (C₁-C₆)alkyl, aryl and aryl(C₁-C₆)alkyl;
R³, R⁴, R⁵, R⁷, R^{3a} and R^{4a} are independently selected from the group consisting of H, (C₁-C₆)alkyl, aryl(C₁-C₆)alkyl, —C(O)(C₁-C₆)alkyl and —C(O)aryl;
R³⁰ is selected from the group consisting of R³²-substituted T, R³²-substituted-T-(C₁-C₆)alkyl, R³²-substituted-(C₂-C₄)alkenyl, R³²-substituted-(C₁-C₆)alkyl, R³²-substituted-(C₃-C₇)cycloalkyl and R³²-substituted-(C₃-C₇)cycloalkyl(C₁-C₆)alkyl;
R³¹ is selected from the group consisting of H and (C₁-C₄)alkyl;
T is selected from the group consisting of phenyl, furyl, thienyl, pyrrolyl, oxazolyl, isoxazolyl, thiazolyl, iosthiazolyl, benzothiazolyl, thiadiazolyl, pyrazolyl, imidazolyl and pyridyl;
R³² is independently selected from 1-3 substituents independently selected from the group consisting of halogeno, (C₁-C₄)alkyl, —OH, phenoxy, —CF₃, —NO₂, (C₁-C₄)alkoxy, methylenedioxy, oxo, (C₁-C₄)alkylsulfanyl, (C₁-C₄)alkylsulfinyl, (C₁-C₄)alkylsulfonyl, —N(CH₃)₂, —C(O)—NH(C₁-C₄)alkyl, —C(O)—N((C₁-C₄)alkyl)₂, —C(O)—(C₁-C₄)alkyl, —C(O)—(C₁-C₄)alkoxy and pyrrolidinylcarbonyl; or R³² is a covalent bond and R³¹, the nitrogen to which it is attached and R³² form a pyrrolidinyl, piperidinyl, N-methyl-piperazinyl, indolinyl or morpholinyl group, or a (C₁-C₄)alkoxycarbonyl-substituted pyrrolidinyl, piperidinyl, N-methylpiperazinyl, indolinyl or morpholinyl group;
Ar¹ is aryl or R¹⁰-substituted aryl;
Ar² is aryl or R¹¹-substituted aryl;
Q is a bond or, with the 3-position ring carbon of the azetidinone, forms the spiro group 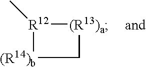
R¹ is selected from the group consisting of
—(CH₂)_{q}—, wherein q is 2-6, provided that when Q forms a spiro ring, q can also be zero or 1;
—(CH₂)ₑ-E-(CH₂)ᵣ—, wherein E is —O—, —C(O)—, phenylene, —NR²²— or —S(O)₀₋₂—, e is 0-5 and r is 0-5, provided that the sum of e and r is 1-6;
—(C₂-C₆)alkenylene-; and
—(CH₂)_{f}—V—(CH₂)_{g}—, wherein V is C₃-C₆ cycloalkylene, f is 1-5 and g is 0-5, provided that the sum of f and g is 1-6;
R¹² is 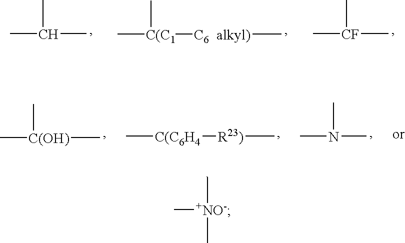
R¹³ and R¹⁴ are independently selected from the group consisting of
—CH₂—, —CH(C₁-C₆ alkyl)-, —C(di-(C₁-C₆)alkyl), —CH═CH— and —C(C₁-C₆ alkyl)=CH—; or R¹² together with an adjacent R¹³, or R¹² together with an adjacent R¹⁴, form a —CH═CH— or a —CH═C(C₁-C₆ alkyl)- group;
a and b are independently 0, 1, 2 or 3, provided both are not zero;
provided that when R¹³ is —CH═CH— or —C(C₁-C₆ alkyl)=CH—, a is 1;
provided that when R¹⁴ is —CH═CH— or —C(C₁-C₆ alkyl)=CH—, b is 1;
provided that when a is 2 or 3, the R¹³'s can be the same or different; and
provided that when b is 2 or 3, the R¹⁴'s can be the same or different;
and when Q is a bond, R¹ also can be: 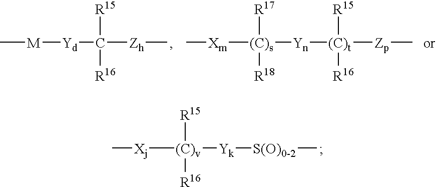
M is —O—, —S—, —S(O)— or —S(O)₂—;
X, Y and Z are independently selected from the group consisting of —CH₂—, —CH(C₁-C₆)alkyl- and —C(di-(C₁-C₆)alkyl);
R¹⁰ and R¹¹ are independently selected from the group consisting of 1-3 substituents independently selected from the group consisting of (C₁-C₆)alkyl, —OR¹⁹, —O(CO)R¹⁹, —O(CO)OR²¹, —O(CH₂)₁₋₅OR¹⁹, —O(CO)NR¹⁹R²⁰, —NR¹⁹R²⁰, —NR¹⁹(CO)R²⁰, —NR¹⁹(CO)OR²¹, —NR¹⁹(CO)NR²⁰R²⁵, —NR¹⁹SO₂R²¹, —COOR¹⁹, —CONR¹⁹R²⁰, —COR¹⁹, —SO₂NR¹⁹R²⁰, S(O)₀₋₂R²¹, —O(CH₂)₁₋₁₀—COOR¹⁹, —O(CH₂)₁₋₁₀CONR¹⁹R²⁰, —(C₁-C₆ alkylene)-COOR¹⁹, —CH═CH—COOR¹⁹, —CF₃, —CN, —NO₂ and halogen;
R¹⁵ and R¹⁷ are independently selected from the group consisting of OR¹⁹, —O(CO)R¹⁹, —O(CO)OR²¹ and —O(CO)NR¹⁹R²⁰;
R¹⁶ and R¹⁸ are independently selected from the group consisting of H, (C₁-C₆)alkyl and aryl; or R¹⁵ and R¹⁶ together are ═O, or R¹⁷ and R¹⁸ together are
d is 1, 2 or 3;
h is 0, 1, 2, 3 or 4;
s is 0 or 1; t is 0 or 1; m, n and p are independently 0-4;
provided that at least one of s and t is 1, and the sum of m, n, p, s and t is 1-6;
provided that when p is 0 and t is 1, the sum of m, s and n is 1-5; and provided that when p is 0 and s is 1, the sum of m, t and n is 1-5;
v is 0 or 1;
j and k are independently 1-5, provided that the sum of j, k and v is 1-5;
when Q is a bond and R¹ is 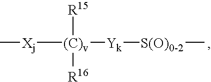
Ar¹ can also be pyridyl, isoxazolyl, furanyl, pyrrolyl, thienyl, imidazolyl, pyrazolyl, thiazolyl, pyrazinyl, pyrimidinyl or pyridazinyl;
R¹⁹ and R²⁰ are independently selected from the group consisting of H, (C₁-C₆)alkyl, aryl and aryl-substituted (C₁-C₆)alkyl;
R²¹ is (C₁-C₆)alkyl, aryl or R²⁴-substituted aryl;
R²² is H, (C₁-C₆)alkyl, aryl(C₁-C₆)alkyl, —C(O)R¹⁹ or —COOR¹⁹;
R²³ and R²⁴ are independently 1-3 groups independently selected from the group consisting of H, (C₁-C₆)alkyl, (C₁-C₆)alkoxy, —COOH, NO₂, —NR¹⁹R²⁰, —OH and halogeno; and
R²⁵ is H, —OH or (C₁-C₆)alkoxy.

11. The pharmaceutical combination according to claim 2, wherein the sterol or 5-α-stanol absorption inhibitor is a compound of formula (IX): 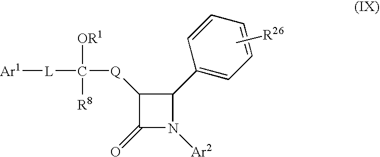
or a pharmaceutically acceptable salt or solvate thereof,
wherein in formula (IX):
R¹ is selected from the group consisting of H, G, G¹, G², —SO₃H and —PO₃H;
G is selected from the group consisting of: H, 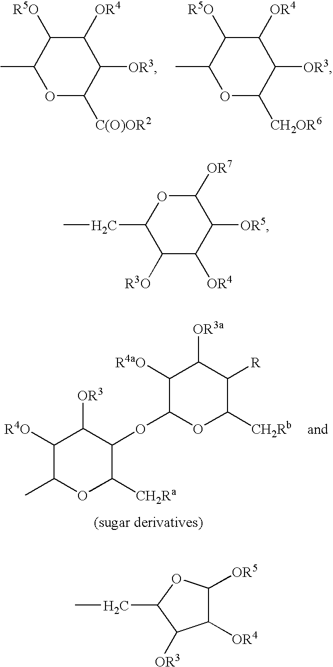
wherein R, R^{a} and R^{b} are each independently selected from the group consisting of H, —OH, halo, —NH₂, azido, (C₁-C₆)alkoxy(C₁-C₆)alkoxy or —W—R³⁰;
W is independently selected from the group consisting of —NH—C(O)—, —O—C(O)—, —O—C(O)—N(R³¹)—, —NH—C(O)—N(R³¹)— and —O—C(S)—N(R³¹)—;
R² and R⁶ are each independently selected from the group consisting of H, (C₁-C₆)alkyl, acetyl, aryl and aryl(C₁-C₆)alkyl;
R³, R⁴, R⁵, R⁷, R^{3a} and R^{4a} are each independently selected from the group consisting of H, (C₁-C₆)alkyl, acetyl, aryl(C₁-C₆)alkyl, —C(O)(C₁-C₆)alkyl and —C(O)aryl;
R³⁰ is independently selected from the group consisting of R³²-substituted T, R³²-substituted-T-(C₁-C₆)alkyl, R³²-substituted-(C₂-C₄)alkenyl, R³²-substituted-(C₁-C₆)alkyl, R³²-substituted-(C₃-C₇)cycloalkyl and R³²-substituted-(C₃-C₇)cycloalkyl(C₁-C₆)alkyl;
R³¹ is independently selected from the group consisting of H and (C₁-C₄)alkyl;
T is independently selected from the group consisting of phenyl, furyl, thienyl, pyrrolyl, oxazolyl, isoxazolyl, thiazolyl, isothiazolyl, benzothiazolyl, thiadiazolyl, pyrazolyl, imidazolyl and pyridyl;
R³² is independently selected from 1-3 substituents which are each independently selected from the group consisting of H, halo, (C₁-C₄)alkyl, —OH, phenoxy, —CF₃, —NO₂, (C₁-C₄)alkoxy, methylenedioxy, oxo, (C₁-C₄)alkylsulfanyl, (C₁-C₄)alkylsulfinyl, (C₁-C₄)alkylsulfonyl, —N(CH₃)₂, —C(O)—NH(C₁-C₄)alkyl, —C(O)—N((C₁-C₄)alkyl)₂, —C(O)—(C₁-C₄)alkyl, —C(O)—(C₁-C₄)alkoxy and pyrrolidinylcarbonyl; or R³² is a covalent bond and R³¹, the nitrogen to which it is attached and R³² form a pyrrolidinyl, piperidinyl, N-methyl-piperazinyl, indolinyl or morpholinyl group, or a (C₁-C₄)alkoxycarbonyl-substituted pyrrolidinyl, piperidinyl, N-methylpiperazinyl, indolinyl or morpholinyl group;
G¹ is represented by the structure: 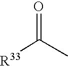
wherein R³³ is independently selected from the group consisting of unsubstituted alkyl, R³⁴-substituted alkyl, (R³⁵)(R³⁶)alkyl-, 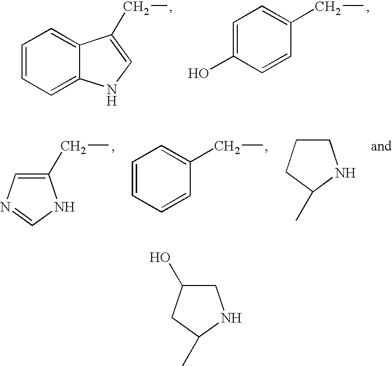
R³⁴ is one to three substituents, each R³⁴ being independently selected from the group consisting of HOOC—, HO—, HS—, (CH₃)S—, H₂N—, (NH₂)(NH)C(NH)—, (NH₂)C(O)— and HOOCCH(NH₃⁺)CH₂SS—;
R³⁵ is independently selected from the group consisting of H and NH₂—;
R³⁶ is independently selected from the group consisting of H, unsubstituted alkyl, R³⁴-substituted alkyl, unsubstituted cycloalkyl and R³⁴-substituted cycloalkyl;
G² is represented by the structure: 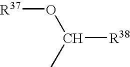
wherein R³⁷ and R³⁸ are each independently selected from the group consisting of (C₁-C₆)alkyl and aryl;
R²⁶ is one to five substituents, each R²⁶ being independently selected from the group consisting of:
a) H;
d) —OH;
e) —OCH₃;
d) fluorine;
e) chlorine;
f) —O-G;
k) —O-G¹;
l) —O-G²;
m) —SO₃H; and
n) —PO₃H;
provided that when R¹ is H, R²⁶ is not H, —OH, —OCH₃ or —O-G;
Ar¹ is aryl, R¹⁰-substituted aryl, heteroaryl or R¹⁰-substituted heteroaryl;
Ar² is aryl, R¹¹-substituted aryl, heteroaryl or R¹¹-substituted heteroaryl;
L is selected from the group consisting of:
f) a covalent bond;
g) —(CH₂)_{q}—, wherein q is 1-6;
h) —(CH₂)ₑ-E-(CH₂)ᵣ—, wherein E is —O—, —C(O)—, phenylene, —NR²²— or —S(O)₀₋₂—, e is 0-5 and r is 0-5, provided that the sum of e and r is 1-6;
i) —(C₂-C₆)alkenylene-;
j) —(CH₂)_{f}—V—(CH₂)_{g}—, wherein V is C₃-C₆cycloalkylene, f is 1-5 and g is 0-5, provided that the sum of f and g is 1-6; and
f) 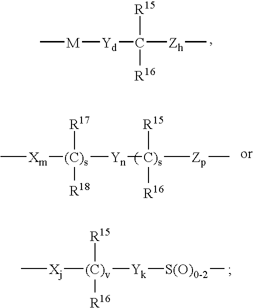
wherein M is —O—, —S—, —S(O)— or —S(O)₂—;
X, Y and Z are each independently selected from the group consisting of —CH₂—, —CH(C₁-C₆)alkyl- and —C(di-(C₁-C₆)alkyl)-;
R⁸ is selected from the group consisting of H and alkyl;
R¹⁰ and R¹¹ are each independently selected from the group consisting of 1-3 substituents which are each independently selected from the group consisting of (C₁-C₆)alkyl, —OR¹⁹, —O(CO)R¹⁹, —O(CO)OR²¹, —O(CH₂)₁₋₅OR¹⁹, —O(CO)NR¹⁹R²⁰, —NR¹⁹R²⁰, —NR¹⁹(CO)R²⁰, —NR¹⁹(CO)OR²¹, —NR¹⁹(CO)NR²⁰R²⁵, —NR¹⁹SO₂R²¹, —COOR¹⁹, —CONR¹⁹R²⁰, —COR¹⁹, —SO₂NR¹⁹R²⁰, S(O)₀₋₂R²¹, —O(CH₂)₁₋₁₀—COOR¹⁹, —O(CH₂)₁₋₁₀CONR¹⁹R²⁰, —(C₁-C₆ alkylene)-COOR¹⁹, —CH═CH—COOR¹⁹, —CF₃, —CN, —NO₂ and halo;
R¹⁵ and R¹⁷ are each independently selected from the group consisting of —OR¹⁹, —OC(O)R¹⁹, —OC(O)OR²¹, —OC(O)NR¹⁹R²⁰;
R¹⁶ and R¹⁸ are each independently selected from the group consisting of H, (C₁-C₆)alkyl and aryl;
or R¹⁵ and R¹⁶ together are ═O, or R¹⁷ and R¹⁸ together are ═O;
d is 1, 2 or 3;
h is 0, 1, 2, 3 or 4;
s is Q or 1;
t is 0 or 1;
m, n and p are each independently selected from 0-4;
provided that at least one of s and t is 1, and the sum of m, n, p, s and t is 1-6; provided that when p is 0 and t is 1, the sum of m, n and p is 1-5; and provided that when p is 0 and s is 1, the sum of m, t and n is 1-5;
v is 0 or 1;
j and k are each independently 1-5, provided that the sum of j, k and v is 1-5;
Q is a bond, —(CH₂)_{q}—, wherein q is 1-6, or, with the 3-position ring carbon of the azetidinone, forms the spiro group
wherein R¹² is 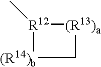 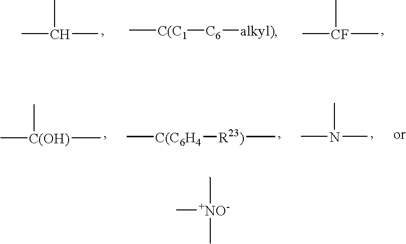
R¹³ and R¹⁴ are each independently selected from the group consisting of —CH₂—, —CH(C₁-C₆ alkyl)-, —C(di-(C₁-C₆)alkyl), —CH═CH— and —C(C₁-C₆ alkyl)=CH—; or R¹² together with an adjacent R¹³, or R¹² together with an adjacent R¹⁴, form a —CH═CH— or a —CH═C(C₁-C₆ alkyl)- group;
a and b are each independently 0, 1, 2 or 3, provided both are not zero; provided that when R¹³ is —CH═CH— or —C(C₁-C₆ alkyl)=CH—, a is 1; provided that when R¹⁴ is —CH═CH— or —C(C₁-C₆ alkyl)-CH—, b is 1; provided that when a is 2 or 3, the R¹³'s can be the same or different; and provided that when b is 2 or 3, the R¹⁴'s can be the same or different;
and when Q is a bond and L is 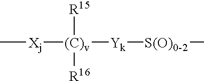
then Ar¹ can also be pyridyl, isoxazolyl, furanyl, pyrrolyl, thienyl, imidazolyl, pyrazolyl, thiazolyl, pyrazinyl, pyrimidinyl or pyridazinyl;
R¹⁹ and R²⁰ are each independently selected from the group consisting of H, (C₁-C₆)alkyl, aryl and aryl-substituted (C₁-C₆)alkyl;
R²¹ is (C₁-C₆)alkyl, aryl or R²⁴-substituted aryl;
R²² is H, (C₁-C₆)alkyl, aryl(C₁-C₆)alkyl, —C(O)R¹⁹ or —COOR¹⁹;
R²³ and R²⁴ are each independently selected from the group consisting of 1-3 substituents which are each independently selected from the group consisting of H, (C₁-C₆)alkyl, (C₁-C₆)alkoxy, —COOH, NO₂, —NR¹⁹R²⁰, —OH and halo; and
R²⁵ is H, —OH or (C₁-C₆)alkoxy.

12. The pharmaceutical combination according to claim 2, wherein the MTP inhibitor is selected from the group consisting of implitapide, mitratapide, T-0126, CP-346086, BMS 201038 and CP-395919.

13. The pharmaceutical combination according to claim 12, wherein the sterol or 5-α-stanol absorption inhibitor is a compound of formula (I): 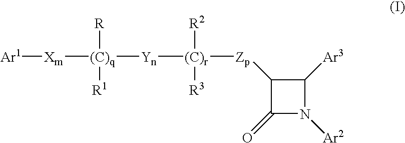
or pharmaceutically acceptable salts or solvates thereof,
wherein in formula (I):
Ar¹ and Ar² are independently selected from the group consisting of aryl and R⁴-substituted aryl;
Ar³ is aryl or R⁵-substituted aryl;
X, Y and Z are independently selected from the group consisting of —CH₂—, —CH(lower alkyl)- and —C(dilower alkyl)-;
R and R² are independently selected from the group consisting of —OR⁶, —O(CO)R⁶, —O(CO)OR⁹ and —O(CO)NR⁶R⁷;
R¹ and R³ are independently selected from the group consisting of hydrogen, lower alkyl and aryl;
q is 0 or 1; r is 0 or 1; m, n and p are independently selected from 0, 1, 2, 3 or 4; provided that at least one of q and r is 1, and the sum of m, n, p, q and r is 1, 2, 3, 4, 5 or 6; and provided that when p is 0 and r is 1, the sum of m, q and n is 1, 2, 3, 4 or 5;
R⁴ is 1-5 substituents independently selected from the group consisting of lower alkyl, —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, -(lower alkylene)COOR⁶, —CH≡CH—COOR⁶, —CF₃, —CN, —NO₂ and halogen;
R⁵ is 1-5 substituents independently selected from the group consisting of —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁶R⁷, —NR⁶SO₂R⁹, —COOR⁶, —CONR⁶R⁷, —COR⁶, —SO₂NR⁶R⁷, S(O)O)₀₋₂R⁹, —O(CH₂)₁₋₁₀—COOR⁶, —O(CH₂)₁₋₁₀CONR⁶R⁷, -(lower alkylene)COOR⁶ and —CH═CH—COOR⁶;
R⁶, R⁷ and R⁸ are independently selected from the group consisting of hydrogen, lower alkyl, aryl and aryl-substituted lower alkyl; and
R⁹ is lower alkyl, aryl or aryl-substituted lower alkyl.

14. The pharmaceutical combination according to claim 13, wherein the sterol or 5-α-stanol absorption inhibitor is ezetimibe.

15. The pharmaceutical combination according to claim 14, which further comprises simastatin.

16. The pharmaceutical combination according to claim 1, which further comprises an effective amount of an HMG-CoA reductase inhibitor.

17. The pharmaceutical combination according to claim 16, wherein the HMG-CoA reductase inhibitor is selected from the group consisting of lovastatin, pravastatin fluvastatin, simvastatin, atorvastatin, cerivastatin, pitavastatin, and rosuvastatin.

18. The pharmaceutical combination according to claim 17, which further comprises an effective amount of an HMG-CoA reductase inhibitor wherein said inhibitor is simvastatin.

19. The pharmaceutical combination according to claim 1, which further comprises a PPAR activator, nicotinic acid and/or a nicotinic acid receptor agonist or a bile acid sequestrant.

20. A method for lipid management in a mammal in need thereof which comprises administering an effective amount of the pharmaceutical combination according to claim 1 to said mammal.

21. The method according to claim 20, wherein the cholesterol absorption inhibitor is ezetimibe and the MTP is selected from the group consisting of implitapide, mitratapide, T-0126, CP-346086, BMS 201038 and CP-395919.

22. The method according to claim 21, wherein the pharmaceutical combination further comprises a cholesterol lowering agent which is a HMG-CoA reductase inhibitor is selected from the group consisting of lovastatin, pravastatin fluvastatin, simvastatin, atorvastatin, cerivastatin, pitavastatin, and rosuvastatin.

23. The method according to claim 22, wherein the HMG Co-A reductase inhibitor is simvastatin.

24. A method for the treatment, prevention or ameliorating the symptoms atherosclerosis in a mammal in need thereof which comprises administering an effective amount of the therapeutic combination according to claim 1 to said mammal.

25. The method according to claim 24, wherein the cholesterol absorption inhibitor is ezetimibe and the MTP is selected from the group consisting of implitapide, mitratapide, T-0126, CP-346086, BMS 201038 and CP-395919.

26. The method according to claim 25, wherein the pharmaceutical combination further comprises a cholesterol lowering agent which is a HMG-CoA reductase inhibitor is selected from the group consisting of lovastatin, pravastatin fluvastatin, simvastatin, atorvastatin, cerivastatin, pitavastatin, and rosuvastatin.

27. The method according to claim 26, wherein the HMG Co-A reductase inhibitor is simvastatin.

28. A method for the prevention or amelioration of the symptoms or the development of hepatic steatosis in a mammal in need thereof comprising administering an effective amount of the pharmaceutical combination according to claim 1 to said mammal.

29. The method according to claim 28, wherein the cholesterol absorption inhibitor is ezetimibe and the MTP is selected from the group consisting of include implitapide, mitratapide, T-0126, CP-346086, BMS 201038 and CP-395919.

30. The method according to claim 29, wherein the pharmaceutical combination further comprises a cholesterol lowering agent which is a HMG-CoA reductase inhibitor is selected from the group consisting of lovastatin, pravastatin fluvastatin, simvastatin, atorvastatin, cerivastatin, pitavastatin, and rosuvastatin.

31. The method according to claim 30, wherein the HMG-CoA reductase inhibitor is simvastatin.
